(19) United States
(12) Patent Application Publication
(10) Pub. No.: US 2023/0045522 A1
(43) Pub. Date: Feb. 9, 2023

(54) COMPOSITIONS AND METHODS TO MODULATE GLUCOSE HOMEOSTASIS

(71) Applicant: The Board of Supervisors of Louisiana State University and Agricultural and Mechanical College, Baton Rouge, LA (US)

(72) Inventor: Elizabeth FLOYD, Baton Rouge, LA (US)

(21) Appl. No.: 17/782,949

(22) PCT Filed: Dec. 3, 2020
(86) PCT No.: PCT/US2020/063055

§ 371 Date: Jun. 6, 2022

Related U.S. Application Data
(60) Provisional application No. 62/944,840, filed on Dec. 6, 2019.

(51) Int. Cl. A61K 36/282; A61K 31/12; A61P 3/10

(52) U.S. Cl. CPC A61K 36/282; A61K 31/12; A61P 3/10

(57) ABSTRACT

This invention is directed to compositions and methods for modulating glucose homeostasis.

This application claims priority from U.S. Provisional Application No. 62/944,840, filed on Dec. 6, 2019, the entire contents of which is incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

### GOVERNMENT INTERESTS

This invention was made with government support under Grant No. P50AT002776 awarded by the National Institutes of Health. The government has certain rights in the invention.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed to compositions and methods for modulating glucose homeostasis.

### BACKGROUND OF THE INVENTION

Type 2 diabetes is a progressive disease characterized by hyperglycemia due to insulin resistance in peripheral tissues coupled with impaired insulin production and increased hepatic glucose output. As insulin resistance is a cornerstone in developing type 2 diabetes, clinical approaches to managing this condition focus on improving peripheral tissue responsiveness to the action of insulin. Although skeletal muscle is the primary site of insulin-mediated glucose disposal, therapeutic strategies targeting skeletal muscle to improve insulin sensitivity have had limited success. Small organic molecules have been tested as potentiators of insulin receptor activation or inhibitors of protein tyrosine phosphatase 1B as approaches to increase insulin sensitivity in skeletal muscle. However, concerns about selectivity, the route of administration, and the risk of hypoglycemia have raised questions about the therapeutic effectiveness of these compounds.

### SUMMARY OF THE INVENTION

The present invention provides compositions and methods for reducing blood glucose levels.

Aspects of the invention are also drawn towards compositions and methods for modulating glucose homeostasis.

Still further, aspects of the invention are drawn towards compositions and methods for treating diabetes.

In embodiments, the methods can comprise administering to a subject in need thereof a therapeutically effective amount of a composition comprising one or more components of PMI-5011.

In embodiments, the one or more components of PMI-5011 function synergistically to reduce blood glucose levels, modulate glucose homeostasis, and/or treat diabetes.

In embodiments, the one or more components can be a methyldavidigenin.

In embodiments, the compositions and methods comprise DMC-2, DMC-1, elemicin, sakuranetin, davidigenin, 6-demethoxycapillarisin or any combination thereof.

In embodiments, the composition and methods comprise KOE.

In embodiments, the one or more components are naturally derived, synthetically produced, or any combination thereof.

Other objects and advantages of this invention will become readily apparent from the ensuing description.

### BRIEF DESCRIPTION OF THE FIGURES

FIG. 1 shows 4-O-Methyldavidigenin (2) is present in skeletal muscle in vitro. C2C12 myotubes were incubated with vehicle control (DMSO) or PMI-5011 (10 μg/mL) for 1, 6, or 16 h as indicated. At each time point, (A) the medium and washes were collected at neutral pH, (B) this was followed by collection of surface bound compounds at acidic pH and cell lysates that were separated into soluble (Lysate) and insoluble fractions (Pellet) for analysis of content by UPLC/MS. (A, B) the mass of 2 recovered in each fraction is shown and (C) the mass balance indicates total 2 was recovered as the parent compound (*, no 6-demethoxycapillarisin; ND, not detected).

FIG. 2 shows UHPLC-UV (A) and ¹H NMR spectroscopic (B) analyses of the PMI-5011 crude extract (in black) vs. the KOE (in red) and the KOF (in green). PMI-5011 and KOE were analyzed at the same concentration of 10 mg/mL for UHPLC-UV and 34 mg/mL for ¹H NMR spectroscopy. The outcomes demonstrate that 2, marked with *, and its regioisomer 1, were removed effectively from the original PMI-5011 crude extract. The KOE preserved the metabolomic profile of the original PMI-5011, except for the removed 2 and 1, as well as the two volatile compounds identified as 3 and 4 (marked with A), for which concentrations were reduced due to inevitable solvent evaporation.

FIG. 3 shows the bioactive marker, 4-O-methyldavidigenin (2) is required for PMI-5011-mediated enhanced AKT phosphorylation. C2C12 or L6 myotubes were incubated with palmitate overnight to induce insulin resistance in the absence (−) or presence (+) of PMI-5011, the extract without 2 (KOE), the removed fraction (KOF), or synthetic 2 at 10 μg/mL each. The cells were harvested after insulin was present for 10 minutes. (A, B) Western blot analysis of total and phosphorylated forms of proteins in insulin signaling pathway. (C) Glycogen content assayed as ng released glucose/μg protein and (D) western blot analysis of GSK3 activation.

FIG. 4 shows experimental design to test metabolic effect of 4-O-methyldavidigenin (2) in vivo. (A) C57BL/6J male mice were fed a 60% kcal fat diet for 16 weeks prior to a 6 h treatment with labrasol (vehicle), metformin, PMI-5011, reconstituted PMI-5011 (RE=KOE+KOF), KOE, KOF, synthetic 2. (B) Body weights at 20 weeks of age for mice randomized to each treatment group. Statistical significance is reported as mean−/+standard deviation, compared to (vehicle) control.

FIG. 5 shows the bioactive marker, 4-O-methyldavidigenin (2) lowers blood glucose levels in a murine model of obesity-induced insulin resistance. (A) Absolute (mg/dL) and (B) percent change in blood glucose levels from baseline for each treatment group six hours after treatment. (C) Insulin levels (ng/mL) and (D) C-Peptide levels (ng/mL) for each treatment group. Statistical significance is reported as mean−/+standard deviation, compared to (vehicle) control; ns, not significant; *p<0.05; ** p<0.01; *** p<0.001.

FIG. 6 shows tissue-specific effect of PMI-5011, KOE and 2 on insulin signaling. (A) Compound 2 enhances AKT phosphorylation and maintains IRO total protein levels in mixed gastrocnemius skeletal muscle. (B) AKT phosphorylation at serine273, but not threonine308 is regulated by 2 in liver, but not (C) in the epididymal adipose tissue of the obese, insulin-resistant male mice.

FIG. 7 shows concentrations of 1 and 2 in the tested extracts and fraction.

FIG. 8 shows a schematic of an embodiment of the disclosure.

FIG. 9 shows CCS step 1: CPC and UHPLC-UV Chromatograms.

FIG. 10 shows CCS step 2: UHPLC-UV chromatograms.

FIG. 11 shows evaluation of sample recovery for KOE and the Reconstituted Extract (RE).

FIG. 12 shows comparative UHPLC-UV chromatograms of PMI-5011 crude extract, KOE and RE.

FIG. 13 shows comparative ¹H NMR spectra of PMI-5011 crude extract, KOE and RE

FIG. 14 shows UHPLC-UV quantitative analysis of (1) and (2) in the different extracts.

FIG. 15 shows UHPLC-UV quantitative analysis of elemicin in PMI-5011 extracts.

FIG. 16 shows annotated ¹H NMR of 4-O-methyldavidigenin and MS/MS spectra of both isomers.

FIG. 17 shows comparative ¹H NMR spectra of KOF with synthetic 4-O-methyldavidigenin.

FIG. 18 shows annotated ¹H NMR of elemicin and MS/MS spectra of both (iso)-elemicin.

FIG. 19 shows UV spectra of methyldavidigenin isomers, and (iso)-elemicin.

FIG. 20 shows antibodies used for western blot analysis.

FIG. 21 shows PMI-5011 activates AMPK-signaling pathway in C2C12 muscle cells and skeletal muscle tissue, but not in liver of mice fed HFD. A-B: Phosphorylation of AMPK at Thr172 and protein expression of AMPKα1 and AMPKα2 in skeletal muscle (A) and liver (B) of mice administered PMI-5011 (500 mg/kg), knockout extract, KOE (500 mg/kg), enriched DMC-2 (100 mg/kg), synthetic DMC-2 at 100 or 300 mg/kg and metformin (300 mg/kg) via gavage (n=3 animals per group). C: Phosphorylation of AMPK at Thr172 and protein expression of AMPKα1 and AMPKα2 in C2C12 myotubes treated with PMI-5011 (10 μg/ml) and DMSO as vehicle for up to 4 hours. D: Phosphorylation of AMPK at Thr172 and AKT at Ser473, and protein expression of AMPKα1, AMPKα2 and total AKT in C2C12 myotubes treated with PMI-5011 (10 μg/ml), synthetic DMC-2 (10 μg/ml), KOE (10 μg/ml) and DMSO as vehicle for up to 16 hours. E: Phosphorylation of AMPK at Thr172 and AKT at Ser473, and protein expression of AMPKα1, AMPKα2 and total AKT in C2C12 muotubes. The myotubes were treated with PMI-5011 (10 μg/ml), KOE (10 μg/ml), enriched DMC-2 (10 μg/ml), synthetic DMC-2 (10 μg/ml), and DMSO as vehicle in the presence of palmitate (200 μM) for 16 hours followed by a 10 minute insulin (200 nM) treatment prior to harvesting cells. Tubulin or β-actin are included as a loading control. Immunoblots shown are representative of three or more independent experiments.

FIG. 22 shows PMI-5011 is more efficient in activating AMPK pathway in muscle cells compared to AICAR and metformin. A: Phosphorylation of AMPK at Thr172 and protein expression of AMPKα1 and AMPKα2 in C2C12 muscle cells treated with PMI-5011 (10 μg/ml), AICAR (0.5 mM), metformin (2 mM) and DMSO as vehicle for up to 16 hours. Tubulin is included as a loading control. B: Comparison of the kinetics of phosphorylation of AMPK at Thr172 in C2C12 muscle cells treated with PMI-5011, AICAR and metformin. Image J software was used for densitometry quantification of the immunoblots in (A). Phosphorylation levels of AMPK at Thr172 were evaluated as ratios of phospho-AMPK to total AMPK, where AMPKα1 and AMPKα2 was combined to calculate total AMPK. The values at 0-time point for each treatment were set to 1 and fold change for each time points was calculated as fold over 0-time for each treatment. C: Phosphorylation levels of AMPK at Thr172 in C2C12 muscle cells treated with PMI-5011, AICAR and metformin at the 16-hr time point. The phosphorylation levels were determined as described in (B) using immunoblot images in (A). Results shown are representative of three independent experiments. Data are means±Std dev; *p<0.05 significance for treatments with AICAR or metformin vs PMI-5011.

FIG. 23 shows AMPK activation by PMI-5011 is LKB1-dependent, but are not CaMKKβ-dependent. A-B: Phosphorylation of LKB1 at Ser428 and protein expression of LKB1 in C2C12 myotubes treated with PMI-5011 (10 μg/ml) (A) and synthetic DMC-2 (10 μg/ml) (B) or DMSO as the vehicle control for up to 16 hours. C: Phosphorylation of LKB1 at Ser428 and protein expression of LKB1 in skeletal muscle of mice administered PMI-5011 (500 mg/kg), knockout extract, KOE (500 mg/kg), enriched DMC-2 (100 mg/kg), synthetic DMC-2 at 100 or 300 mg/kg, and metformin (300 mg/kg body weight) via gavage (n=3 animals per group). Tubulin or β-actin are included as a loading control. D: Phosphorylation of AMPK at Thr172 and protein expression of AMPKα1 and AMPKα2 in C2C12 myotubes treated with PMI-5011 (10 μg/ml), synthetic DMC-2 (10 μg/ml) and KOE (10 μg/ml) in the presence of FK-506 (10 μM) or STO-609 (10 μg/ml) for 16 hours. Results shown are representative of three or more independent experiments.

FIG. 24 shows downstream effectors of AMPK signaling are regulated by PMI-5011. A: Phosphorylation of ACC at Ser79 and protein expression of ACC in C2C12 myotubes treated with PMI-5011 (10 μg/ml), KOE (10 μg/ml), enriched DMC-2 (10 μg/ml), synthetic DMC-2 (10 μg/ml) in the presence of palmitate (200 μM) for 16 hours, followed by insulin (200 nM) for 10 minutes. B: Protein expression of p-ACC, ACC and SIRT1 in skeletal muscle of mice administered PMI-5011 (500 mg/kg), KOE (500 mg/kg), enriched DMC-2 (100 mg/kg), synthetic DMC-2 at 100 or 300 mg/kg and metformin (300 mg/kg) via gavage (n=3 animals per group). β-actin is included as a loading control.

FIG. 25 shows PMI-5011 acts on both AMPKα1 and AMPKα2 by enhancing their phosphorylation at Thr172 in the cytoplasm and nucleus. A: Phosphorylation of AMPK at Thr172 and protein expression of AMPKα1 and AMPKα2 in C2C12 myotubes in the presence of siRNA for AMPKα1 or AMPKα2 and non-targeting (NT) control. B: Phosphorylation of AMPK at Thr172 and protein expression of AMPKα1 and AMPKα2 in C2C12 myotubes treated with PMI-5011 (10 μg/ml) and DMSO as vehicle in the presence of siRNA for AMPKα1 or AMPKα2 and non-targeting (NT) control for 48 hours. C. Cytoplasmic and nuclear AMPK phosphorylation at Thr172 is increased by PMI-5011. Tubulin is a marker of total lysate and the cytoplasmic fraction; histones mark the nuclear fraction.

FIG. 26 shows PMI-5011 and KOE enhance phosphorylation of AMPK despite proteasome-dependent reduction of protein expression of AMPKα1 or AMPKα2 in muscle cells. A-C: Phosphorylation of AMPK at Thr172 and protein expression of AMPKα1, AMPKα2 and LKB1 in C2C12 muscle cells treated with PMI-5011 (10 μg/ml) (A), KOE (10 μg/ml) (B) and synthetic DMC-2 (10 μg/ml) (C) in the absence or presence of MG132 (10 μM) for up to 4 hours. Tubulin is included as a loading control. Results shown are representative of three independent experiments.

FIG. 27 shows schematic presentation of PMI-5011 action on AMPK-signaling pathway in muscle cells. A: Main outcomes of AMPK activation by PMI-5011 at the cellular level. B: Model of mechanisms of activation of AMPK by PMI-5011; allosterically activating by direct binding toy subunit of AMPK (point 1), promoting Thr172-phosphorylation of a subunit of AMPK by LKB1 (point 2), or inhibiting Thr172-dephosphorylation of a subunit of AMPK by blocking protein phosphatases (PP) access to Thr172 site (point 3).

FIG. 28 shows AKT and AMPK are reciprocally activated in skeletal muscle under basal conditions. Western blot analysis of AKT phosphorylation at serine 473 and threonine 308 relative to total AKT and AMPK phosphorylation at threonine 172 relative to AMPK alpha 1 and AMPK alpha 2 isoforms. The protein levels and phosphorylation levels are measured in C2C12 myotubes under basal conditions over a 16-hour period as shown.

### DETAILED DESCRIPTION OF THE INVENTION

Type 2 diabetes is characterized by hyperglycemia due to insulin resistance and impaired insulin production. The extract, PMI-5011, reduces blood glucose levels and improves insulin levels in mouse models of obesity-induced insulin resistance. PMI-5011 enhances insulin signaling in mouse and human skeletal muscle cells. DMC-2 and DMC-1 are important components of PMI-5011. For example, purified DMC-2 has a pronounced hypoglycemic effect by reducing blood glucose levels within 6 hours of administration to obese mice. DMC-2 stimulates phosphorylation of protein kinase B (AKT), a critical factor in insulin signaling.

As described herein the role of DMCs were investigated using a DMC-knockout equivalent (DMC-KOE) of PMI-5011, rather than investigating the role of each of the purified compounds found in PMI-5011. The prior methods (e.g., BFG) fail to capture the therapeutic potential related to compound interactions in complex mixtures such as PMI-5011. The methods utilized herein address this, and essentially permit the investigators to knockout the previously identified DMC-2 and DMC-1 and compare the activities of the knockout with intact PMI-5011, a mixture of DMC-2 and DMC-1, and synthetic DMC-2.

As shown herein, DMCs are involved in glucose homeostasis and insulin signaling. DMC-2 stimulates phosphorylation of AKT. The steady-state levels of several proteins are differentially regulated by DMC-2 and DMC-KOE. DMC-2 appears to be more effective at lower concentrations when embedded within the PMI-5011 matric than when administered as a purified compound.

Detailed descriptions of one or more embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein, the term “about” can refer to approximately, roughly, around, or in the region of. When the term “about” is used in conjunction with a numerical range, it modifies that range by extending the boundaries above and below the numerical values set forth. The term “about” can refer to modifing a numerical value above and below the stated value by a variance of 20 percent up or down (higher or lower).

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges can independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as understood by one of ordinary skill in the art to which this disclosure belongs. Although any methods and materials similar or equivalent to those described herein can also be used in the practice or testing of the present disclosure, the methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for.

Before the embodiments of the present disclosure are described in detail, unless otherwise indicated, the present disclosure is not limited to particular materials, reagents, reaction materials, manufacturing processes, or the like, as such can vary. For example, the terminology used herein is for purposes of describing embodiments only and is not intended to be limiting. For example, the steps can be executed in different sequence where this is logically possible.

Therapeutic Compositions

Aspects of the invention are drawn towards therapeutic compositions and uses thereof that comprise one or more components of the botanical extract PMI5011.

In embodiments, the phrases “therapeutic composition,” “pharmaceutical compositions”, and “botanical extract” can be used interchangeably.

A “botanical extract” can refer to a fresh or processed (e.g. cleaned, frozen, dried, sliced, liquified) part of a single species of plant or a fresh or processed alga or macroscopic fungus. For example, the botanical extract is a plant extract. In embodiments, the botanical extract can be isolated from Artemisia spp., such as Artemisia dracunculus L.

In embodiments, the botanical extract can be an isolated extract. An “isolated extract” can refer to one or more compounds present in or obtained from a plant, such as Artemisia dracunculus L. In an embodiment, the extract can refer to a mixture or blend of compounds present in the plant. Such compound(s) can be obtained by extracting a whole or part of a plant. The extraction step can be optionally followed by further enrichment steps. The terms “extract” and “isolated extract” can be used interchangeably.

In an embodiment, the extract comprises an alcoholic extract (Ribnicky et al. 2006, Phytomedicine, 13, 550; Longendra et al. 2006, Phytochemistry, 67, 1539; Schmidt et al. 2008, Metabolism, 57, S3; and Ribnicky et al. 2009, Int. J. Pharm., 370, 87). For example, the extract comprises an ethanolic extract. In another embodiment, the extract comprises a polar extract. For example, the extract comprises ethyl acetate. For example, the extract comprises hot water.

In embodiments, the isolated extract comprises PMI5011 or an isolated component thereof. For example, “PMI5011” can refer to the ethanolic botanical extract of Artemisia dracunculus L. For example, the isolated extract comprises less than about 0.01% PMI5011, about 0.01% PMI5011, about 0.05% PMI5011, about 0.10% PMI5011, about 0.15% PMI5011, about 0.20% PMI5011, about 0.25% PMI5011, about 0.30% PMI5011, about 0.40% PMI5011, about 0.50% PMI5011, about 1.0% PMI5011, about 1.5% PMI5011, about 2.0% PMI5011, about 2.5% PMI5011, about 3.0% PMI5011, about 3.5% PMI5011, about 4.0% PMI5011, about 4.5% PMI5011, about 5% PMI5011, about 7.5% PMI5011, about 10% PMI5011, or greater than about 10% PMI5011.

Embodiments herein can comprise a component isolate from a botanical extract from Artemisia spp., such as Artemisia dracunculus L. An “isolated component thereof” can refer to any compound or mixture of compounds that is isolated from a plant extract or botanical extract. The isolated component can be a single compound, a homologous mixture or blend of similar compounds, or a heterologous mixture of dissimilar compounds. For example, the isolated component comprises an isolated component of PMI5011 thereof. Other non-limiting examples of isolated components comprises DMC-1, DMC-2, davidigenin, sakuranetin, or 6-demethoxycappillarisin. Embodiments can comprise combinations of isolated components, including those described herein.

In embodiments, the isolated extract can comprise a knockout extract (KOE). A “knockout extract” can refer to an extract which contains all components of an extract except for at least one target compound. A knockout extract can reveal the effects of bioactive compounds in a crude extract. In some embodiments, the KOE is deficient in at least one of DMC-1, DMC-2, davidigenin, sakuranetin, or 6-demethoxylcapillarisin. In some embodiments, the KOE is deficient in DMC-1 and DMC-2. In some embodiments, the KOE is deficient in a combination of components. See, for example, Yu, Yongmei, et al. “The DESIGNER Approach Helps Decipher the Hypoglycemic Bioactive Principles of Artemisia dracunculus (Russian Tarragon)” Journal of Natural Products (2019), which is incorporated by reference herein in its entirety.

A “therapeutic composition” can refer to a composition comprising one or more active ingredient(s) required to cause a desired effect when an effective amount of the composition is administered to a subject in need thereof. For example, the desired effect can be prevention or treatment of a metabolic disorder, such as diabetes, lowering blood sugar, and/or regulating glucose homeostasis in a subject.

In embodiments, the active ingredients of the therapeutic composition can be synthetically produced. The term “synthetically produced” can refer to the production of compounds, such as those described herein, using synthetic techniques well known to those skilled in the art for the purpose of obtaining such compounds. In embodiments, the components of the compositions can be “naturally produced” or are “naturally occurring”. “Naturally produced” can refer to that available from animal or plant sources. In embodiments, the naturally produced active ingredients can be modified in a manner such that they are no longer natural products.

The term “pharmaceutically acceptable” can refer to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” can mean a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The pharmaceutical carrier or diluent employed can be a conventional solid or liquid carrier. Examples of solid carriers are lactose, terra alba, sucrose, cyclodextrin, talc, gelatin, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene and water. Similarly, the carrier or diluent can include any sustained release material known in the art, such as glyceryl monostearate or glyceryl distearate, alone or mixed with a wax.

When a solid carrier is used for oral administration, the preparation can be tabletted, placed in a hard gelatin capsule in powder or pellet form or it can be in the form of a troche or lozenge. The amount of solid carrier can vary widely but can be from about 25 mg to about 1 g.

When a liquid carrier is used, the preparation can be in the form of a syrup, emulsion, soft gelatin capsule or sterile injectable liquid such as an aqueous or non-aqueous liquid suspension or solution.

The compositions disclosed herein can be used therapeutically in combination with a pharmaceutically, nutraceutically, or cosmetically acceptable carrier. Suitable carriers and their formulations are described in Remington: The Science and Practice of Pharmacy (19th ed.) ed. A. R. Gennaro, Mack Publishing Company, Easton, Pa. 1995. An appropriate amount of a pharmaceutically-acceptable salt is used in the formulation to render the formulation isotonic. Examples of the pharmaceutically-acceptable carrier include, but are not limited to, saline, Ringer's solution, and dextrose solution. The pH of the solution is from about 5 to about 8, for example, from about 7 to about 7.5.

Compositions can also include carriers, thickeners, diluents, buffers, preservatives, surface active agents and the like in addition to the molecule of choice. According to the invention, a pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

Embodiments described herein can be provided in a therapeutically effective amount. The term “therapeutically effective amount” can refer to the amount of the composition used is of sufficient quantity to ameliorate one or more causes or symptoms of a disease or disorder. Such amelioration only requires a reduction or alteration, not necessarily elimination. For example, compositions as described herein can comprise therapeutically effective amounts of one or more components of PMI-5011, an ethanolic extract of Artemisia dracunculus L. For example, the component can be a methyldavidigenin, such as 2′,4′-dihydroxy-4-methoxydihydrochalcone (DMC-2), 4,2′-dihydroxy-4′-methoxydihydrochalcone (DMC-1). Compositions can further comprise one or more additional components, such as elemicin, sakuranetin, davidigenin, 6-demethoxycapillarisin. In embodiments, the composition comprises one or more components of PMI-5011, wherein the components are not DMC-1 and/or DMC-2.

The therapeutically effective amount can vary depending upon a number of factors known to those of ordinary skill in the art. For example, the therapeutically effective amount can vary depending upon the identity, age, sex, health, weight, size, and condition of the subject or sample being treated, and the nature and extent of the condition. The therapeutically effective amount can further depend on the effect which is desired by the practitioner, pharmacodynamic characteristics of the active ingredient and its mode and route of administration; time of administration of active ingredient; kind of concurrent treatment, frequency of treatment and the effect desired; and rate of excretion. These amounts can be readily determined by the skilled artisan. For example, a therapeutically effective amount can be an amount of a composition described herein needed to treat diabetes, lower blood sugar or maintain glucose homeostasis.

In embodiments, a therapeutically effective amount can comprise a dose of about 0.005 mg/kg to about 1000 mg/kg. In some embodiments, a therapeutically effective amount can comprise a dose of about 0.005 mg/kg to about 10 mg/kg. In some embodiments, a therapeutically effective amount can comprise a dose of about 0.25 mg/kg to about 2 mg/kg. In some embodiments, the therapeutically effective amount is at least about 0.001 mg/kg at least about 0.0025 mg/kg, at least about 0.005 mg/kg, at least about 0.01 mg/kg, at least about 0.1 mg/kg body weight, at least about 0.25 mg/kg body weight, at least about 0.5 mg/kg body weight, at least about 0.75 mg/kg body weight, at least about 1 mg/kg body weight, at least about 2 mg/kg body weight, at least about 3 mg/kg body weight, at least about 4 mg/kg body weight, at least about 5 mg/kg body weight, at least about 6 mg/kg body weight, at least about 7 mg/kg body weight, at least about 8 mg/kg body weight, at least about 9 mg/kg body weight, at least about 10 mg/kg body weight, at least about 15 mg/kg body weight, at least about 20 mg/kg body weight, at least about 25 mg/kg body weight, at least about 30 mg/kg body weight, at least about 40 mg/kg body weight, at least about 50 mg/kg body weight, at least about 75 mg/kg body weight, at least about 100 mg/kg body weight, at least about 200 mg/kg body weight, at least about 250 mg/kg body weight, at least about 300 mg/kg body weight, at least about 3500 mg/kg body weight, at least about 400 mg/kg body weight, at least about 450 mg/kg body weight, at least about 500 mg/kg body weight, at least about 550 mg/kg body weight, at least about 600 mg/kg body weight, at least about 650 mg/kg body weight, at least about 700 mg/kg body weight, at least about 750 mg/kg body weight, at least about 800 mg/kg body weight, at least about 900 mg/kg body weight, or at least about 1000 mg/kg body weight. However, the skilled artisan will recognize that the dosage can vary depending upon known factors such as the pharmacodynamic characteristics of the active ingredient and its mode and route of administration; time of administration of active ingredient; age, sex, health and weight of the recipient; nature and extent of symptoms; kind of concurrent treatment, frequency of treatment and the effect desired; and rate of excretion.

Embodiments of the invention can be provided as a pharmaceutically acceptable salt. The term “pharmaceutically acceptable” can refer to salts or chelating agents are acceptable from a toxicity viewpoint. The term “pharmaceutically acceptable salt” can refer to refer to ammonium salts, alkali metal salts such as potassium and sodium (including mono, di- and tri-sodium) salts, alkaline earth metal salts such as calcium and magnesium salts, salts with organic bases such as dicyclohexylamine salts, N-methyl-D-glucamine, and salts with amino acids such as arginine, lysine, and so forth.

A pharmaceutical composition of the invention is formulated to be compatible with its intended route of administration. Such compositions can comprise, for example, the active ingredient(s) and a pharmaceutically acceptable carrier. Such compositions can be in a form adapted to oral, subcutaneous, parenteral (intravenous, intraperitoneal), intramuscular, rectal, epidural, intratracheal, intranasal, dermal, vaginal, buccal, ocularly, or pulmonary administration, such as in a form adapted for administration by a peripheral route or is suitable for oral administration or suitable for parenteral administration. Other routes of administration are subcutaneous, intraperitoneal and intravenous, and such compositions can be prepared in a manner well-known to the person skilled in the art, e.g., as described in “Remington's Pharmaceutical Sciences”, 17. Ed. Alfonso R. Gennaro (Ed.), Mark Publishing Company, Easton, Pa., U.S.A., 1985 and more recent editions and in the monographs in the “Drugs and the Pharmaceutical Sciences” series, Marcel Dekker. The compositions can appear in conventional forms, for example, solutions and suspensions for injection, capsules and tablets, for example in the form of enteric formulations, e.g. as disclosed in U.S. Pat. No. 5,350,741, for oral administration.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, and thimerosal. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the compound in the required amount in an appropriate solvent with one or a combination of ingredients enumerated herein, as required, followed by filtered sterilization. Dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

Oral compositions can include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as known in the art.

In embodiments, the composition can be prepared as a medical food, dietary item or food ingredient. The term “dietary item” can include any product that undergoes at least one processing or culinary step prior to distribution and is consumed by a subject. Non-limiting examples of processing and culinary steps include mixing, cooking, baking, heating, chopping, chilling, freezing, packaging, canning, bagging, and storing. Non-limiting examples of dietary items include food products, dietary ingredients, medical foods, functional foods, beverages, dietary supplements, vitamins, minerals, and combinations thereof. Unprocessed, raw, or fresh foods, such as fresh fruits and vegetables, are not included herein within this term.

The term “food ingredient” can refer to any edible substance that is combined is with other edible substances, where the final combination is consumed as a food. The term “medical food” herein is defined by statute in the United States of America, Orphan Drug Act, section 5(b) (21 U.S.C. 360ee (b) (3)), which defines “medical food” as “a food which is formulated to be consumed or administered enterally under the supervision of a physician and which is intended for the specific dietary management of a disease or condition for which distinctive nutritional requirements, based on recognized scientific principles, are established by medical evaluation.”

Many embodiments of the invention are suitable for topical administration to a subject. Non-limiting examples of such embodiments comprise solutions, lotions, creams, ointments, gels, pastes, sprays, liquids, washes, hydrating agents or solutions, and perfusing agents or solutions. Topical doses of a compositions is higher than those doses if administered orally or intravenously, for example, as getting across the skin often requires a higher dose.

The term “combination” can refer to either a fixed combination in one dosage unit form, or a kit of parts for the combined administration where a compound and a combination partner (e.g., another drug, also referred to as “therapeutic agent” or “co-agent”) can be administered independently at the same time or separately within time intervals, especially where these time intervals allow that the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” can refer to a product that results from the mixing or combining of more than one active ingredient and includes both fixed and non-fixed combinations of the active ingredients. The term “fixed combination” can refer to active ingredients, e.g., a compound and a combination partner, both administered to a patient simultaneously in the form of a single entity or dosage. The term “non-fixed combination” can refer to active ingredients, e.g., a compound and a combination partner, that are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

For example, the term “combination” can refer to a pharmaceutical composition comprising a first agent and a second agent, for example. The term “agent”, such as “first agent” or “second active agent”, can be used to describe and is intended to include the formulation according to the present invention. The second active agent can be different from the first active agent. For example, the first agent can be DMC-2 and the second agent can be DMC-1, elemicin, sakuranetin, davidigenin, 6-demethoxycapillarisin, or another component, such as of PMI-5011, or vice versa. It will be recognized that one or more additional active agents can optionally be included in the formulation.

In one embodiment, the pharmaceutical composition can comprise 100% of a first agent, such as 100% DMC-2. In other embodiments, the pharmaceutical composition can comprise about 100% of a first agent, about 90% of a first agent, about 80% of a first agent, about 70% of a first agent, about 60% of a first agent, about 50% of a first agent, about 40% of a first agent, about 30% of a first agent, about 20% of a first agent, about 10% of a first agent, or about 1% of a first agent.

In an embodiment, the pharmaceutical composition can be in ranges of 99:1 to 0:100 first agent:second agent, and up to 100:0 to 0:100 first agent:second agent. For example, the pharmaceutical composition can be about 99:1 first agent:second agent, about 90:10 first agent:second agent, about 80:20 first agent:second agent, about 70:30 first agent: second agent, about 60:40 first agent:second agent, about 50:50 first agent:second agent, about 40:60 first agent:second agent, about 30:70 first agent:second agent, about 20:80 first agent:second agent, about 10:90 first agent:second agent, or about 0:100 first agent:second agent.

Methods of Treatment

Aspects of the invention are drawn towards methods of reducing blood glucose levels in a subject, methods of modulating glucose homeostasis in a subject, or methods of treating a subject afflicted with diabetes.

The term “subject” can refer to any individual who is the target of administration or treatment, e.g., for experimental, diagnostic, prophylactic, and/or therapeutic purposes. The subject can be a vertebrate, for example, a mammal. Thus, the subject can be a human or veterinary patient. For veterinary applications, a wide variety of subjects will be suitable, e.g., livestock such as cattle, sheep, goats, cows, swine, and the like; poultry such as chickens, ducks, geese, turkeys, and the like; and domesticated animals for example, pets such as dogs and cats. For diagnostic or research applications, a wide variety of mammals will be suitable subjects, including rodents (e.g., mice, rats, hamsters), rabbits, primates, and swine such as inbred pigs and the like. The term “patient” can refer to a subject under the treatment of a clinician, e.g., physician. The term “living subject” refers to a subject noted above or another organism that is alive. The term “living subject” refers to the entire subject or organism and not just a part excised (e.g., a liver or other organ) from the living subject.

The term “treatment” can refer to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “inhibit” can refer to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

Aspects of the invention are drawn towards treating or preventing a metabolic disorder. The term “metabolic disorder” can refer to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 in the brain), or the like. Examples of metabolic disorders include, but are not limited to, diabetes (e.g., type 1 diabetes, type 2 diabetes, gestational diabetes), hyperglycemia, hyperinsulinemia, insulin resistance, metabolic syndrome, and obesity.

The term “metabolic syndrome” can refer to a cluster of metabolic abnormalities including abdominal obesity, insulin resistance, glucose intolerance, diabetes, hypertension and dyslipidemia. These abnormalities are known to be associated with an increased risk of vascular events.

The term “diabetes,” as used herein, includes both insulin-dependent diabetes mellitus (i.e., IDDM, also known as type 1 diabetes) and non-insulin-dependent diabetes mellitus (i.e., NIDDM, also known as Type 2 diabetes). Type 1 diabetes, or insulin-dependent diabetes, is the result of an absolute deficiency of insulin, the hormone which regulates glucose utilization. Type 2 diabetes, or insulin-independent diabetes (i.e., non-insulin-dependent diabetes mellitus), often occurs in the face of normal, or even elevated levels of insulin and appears to be the result of the inability of tissues to respond appropriately to insulin. Most of the Type 2 diabetics are also obese. The compositions of the present disclosure are useful for treating both Type 1 and Type 2 diabetes. The compositions are especially effective for treating Type 2 diabetes. The compositions described herein are also useful for treating and/or preventing gestational diabetes mellitus.

Treatment of diabetes mellitus can refer to the administration of a compound or combination of the present invention to treat diabetes. One outcome of treatment can be decreasing the glucose level in a subject with elevated glucose levels. Another outcome of treatment can be decreasing insulin levels in a subject with elevated insulin levels. Another outcome of treatment is decreasing plasma triglycerides in a subject with elevated plasma triglycerides. Another outcome of treatment is decreasing LDL cholesterol in a subject with high LDL cholesterol levels. Another outcome of treatment is increasing HDL cholesterol in a subject with low HDL cholesterol levels. Another outcome of treatment is increasing insulin sensitivity. Another outcome of treatment may be enhancing glucose tolerance in a subject with glucose intolerance. Yet another outcome of treatment may be decreasing insulin resistance in a subject with increased insulin resistance or elevated levels of insulin.

Prevention of diabetes mellitus refers to the administration of a compound or combination described herein to prevent the onset of diabetes in a subject in need thereof.

The term “administration” can refer to introducing a substance, such as a composition or a botanical extract, into a subject. For example, any route of administration may be utilized including, for example, those described herein.

In embodiments, administering can comprise the placement of a pharmaceutical composition, such as a composition comprising a botanical extract, into a subject by a method or route which results in at least partial localization of the composition at a desired site such that desired effect is produced.

For example, the pharmaceutical composition can be administered by bolus injection or by infusion. A bolus injection can refer to a route of administration in which a syringe is connected to the IV access device and the medication is injected directly into the subject. The term “infusion” can refer to an intravascular injection.

Embodiments as described herein can be administered to a subject one time (e.g., as a single injection, bolus, or deposition). Alternatively, administration can be once or twice daily to a subject for a period of time, such as from about 2 weeks to about 28 days. It can also be administered once or twice daily to a subject for period of 1, 2, 3, 4, 5, 6, 7, 8, 9, 10, 11, 12 times per year, or a combination thereof.

In embodiments, compositions as described herein can be administered to a subject chronically. “Chronic administration” can refer to administration of the botanical extract in a continuous manner, such as to maintain the therapeutic effect (activity) over a prolonged period of time.

In an embodiment, the extract is administered to a subject orally, intravenously, sub-cutaneously, or transdermally. For example, the subject is administered less than 1 μg of extract, about 1 μg of extract, about 2 μg of extract, about 5 μg of extract, about 10 μg of extract, about 50 μg of extract, about 1 mg of extract, about 5 mg of extract, about 10 mg of extract, about 50 mg of extract, about 100 mg of extract, about 500 mg of extract, about 1 g of extract, or greater than 1 g of extract.

Disclosed herein is a method of modulating glucose homeostasis, reducing blood glucose levels, and treating diabetes in a subject, comprising administering to a subject in need thereof a therapeutically effective amount of a composition described herein.

For example, an aspect of the invention is drawn to methods of reducing blood glucose levels in a subject. For example, an embodiment can comprise administering to a subject in need thereof a therapeutically effective amount of a composition as described herein. As used herein, “reduce” or “reducing” blood glucose can refer to a decrease in the amount of blood glucose observed or measured in the blood of a subject after administration of a composition as described herein. For example, blood glucose levels can be reduced by about 10%, about 20%, about 30%, about 40%, about 50%, about 60%, about 70%, about 80%, about 90%, or about 100%.

Another aspect of the invention is drawn to methods of modulating glucose homeostasis in a subject. The term “glucose homeostasis” can refer to the balance of insulin and glucagon to maintain blood glucose levels within normal levels (i.e about 64.8 to 104.4 mg/dL). A composition that “promotes glucose hemostasis” or “modulates glucose homeostasis” can reduces blood glucose levels down to, but not below, normal levels.

A further aspect of the invention is drawn to methods of treating a subject afflicted with diabetes. “Diabetes” can refer to a heterogeneous group of disorders that share impaired glucose intolerance, hyperglycemia, and/or insulin resistance in common. Type I diabetes can be characterized by pancreatic endocrine insufficiency or absence; and type II can be characterized by insulin resistance. Diabetes can refer to disorders in which carbohydrate utilization is reduced; and can be characterized by hyperglycemia, glycosuria, ketoacidosis, neuropathy or nephropathy, increased hepatic glucose production, insulin resistance in various tissues, insufficient insulin secretion and enhanced or poorly controlled glucagon secretion from the pancreas.

Kits

Aspects of the invention are directed towards kits for preventing or treating a metabolic disease, such as diabetes.

The term “kit” can refer to a set of articles that facilitates the process, method, assay, analysis, or manipulation of a sample. The kit can include instructions for using the kid (eg, instructions for the method of the present invention), materials, solutions, components, reagents, chemicals, or enzymes required for the method, and other optional components.

The botanical extract and/or compositions as described herein can be provided in a kit. In one embodiment, the kit includes (a) a container that contains a composition that includes a botanical extract or components thereof, and optionally (b) informational material. The informational material can be descriptive, instructional, marketing or other material that relates to the methods described herein and/or the use of the agents for therapeutic benefit. In an embodiment, the kit also includes a second agent, such as a second agent for treating a metabolic disease or disorder. For example, the kit includes a first container that contains the botanical extract or composition comprising the same, and a second container that includes the second agent.

The informational material of the kits is not limited in its form. In one embodiment, the informational material can include information about production of the compound, molecular weight of the compound, concentration, date of expiration, batch or production site information, and so forth. In one embodiment, the informational material relates to methods of administering the botanical extract, e.g., in a suitable dose, dosage form, or mode of administration (e.g., a dose, dosage form, or mode of administration described herein), to treat a subject). The information can be provided in a variety of formats, include printed text, computer readable material, video recording, or audio recording, or a information that provides a link or address to substantive material.

In addition to the botanical extract, the composition in the kit can include other ingredients, such as a solvent or buffer, a stabilizer, or a preservative. The botanical extract or components thereof can be provided in any form, e.g., liquid, dried or lyophilized form, and can be substantially pure and/or sterile. When the botanical extract is are provided in a liquid solution, the liquid solution can be an aqueous solution or an alcohol solution. When the botanical extract or components thereof are provided as a dried form, reconstitution is by the addition of a suitable solvent. The solvent, e.g., sterile water or buffer, can optionally be provided in the kit.

The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., a unit that includes the botanical extract and the second agent, e.g., in a desired ratio. For example, the kit includes a plurality of syringes, ampules, foil packets, blister packs, or medical devices, e.g., each containing a single combination unit dose. The containers of the kits can be air tight, waterproof (e.g., impermeable to changes in moisture or evaporation), and/or light-tight. The kit optionally includes a device suitable for administration of the composition, e.g., a syringe or other suitable delivery device. The device can be provided pre-loaded with one or both of the agents or can be empty, but suitable for loading.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Regulation of Glucose Metabolism by 2′,4′-dehydroxy-4-methoxydihydrochalcone

An ethanolic extract from Russian tarragon, termed PMI5011 can enhance insulin signaling in skeletal muscle and improve systemic insulin sensitivity and glucose homeostasis. Bioactivity-guided studies identified 2′,4′-dihydroxy-4-methoxydihydrochalcone (“DMC-2”) as having bioactivity related to glucose homeostasis. Using the total extract (PMI5011), a form of the extract devoid of DMC-2 and its isomer, the DMC-2 enriched fraction, synthesized DMC-2 or metformin, we carried out assays of insulin signaling using C2C12 myotubes as an in vitro model of insulin resistance and in vivo in a rodent model of obesity-induced insulin resistance. The total extract, the enriched DMC-2 and synthesized DMC-2 stimulate insulin signaling in vitro and in vivo. Specifically, DMC-2 stimulates phosphorylation of protein kinase B (AKT), a critical factor in insulin signaling. Moreover, the in vivo studies show that DMC-2 reduced blood glucose levels comparable to Metformin within 6 hours of oral gavage. This correlates with enhanced insulin signaling in skeletal muscle, but not adipose tissue or liver, indicating skeletal muscle is the primary site of action.

### Example 2

The DESIGNER Approach Helps Decipher the Hypoglycemic Bioactive Principles of Artemisia dracunculus (Russian Tarragon)

Abstract: Complementing classical drug discovery, phytochemicals act on multiple pharmacological targets, especially in botanical extracts where they form complex bioactive mixtures. The reductionist approach used in bioactivity-guided fractionation to identify single bioactive phytochemicals is inadequate for capturing the full therapeutic potential of the (bio)chemical interactions present in such complex mixtures. This study used a DESIGNER (Deplete and Enrich Select Ingredients to Generate Normalized Extract Resources) approach to selectively remove the known bioactives, 4′-O-methyldavidigenin (1; 4,2′-dihydroxy-4′-methoxydihydrochalcone, syn. DMC-1) and its isomer 4-O-methyldavidigenin (2; syn. DMC-2), from the mixture of phytochemicals in an ethanol extract from Artemisia dracunculus to determine, to what degree the more abundant 2 accounts for the established antidiabetic effect of the A. dracunculus extract. Using an otherwise chemically intact “knock-out extract” depleted in 2 and its regioisomer, 1, in vitro and in vivo outcomes confirmed that 2 (and likely 1) act as major bioactive(s) that enhance(s) insulin signaling in skeletal muscle, but also revealed that 2 does not account for the breadth of detectable biological activity of the extract. This is the first report of generating, at a sufficiently large preparative scale, a “knock-out extract” used as a pharmacological tool for in vitro and in vivo studies to dissect the biological impact of a bioactive in a complex phytochemical mixture.

Introduction: Type 2 diabetes is a progressive disease characterized by hyperglycemia due to insulin resistance in peripheral tissues coupled with impaired insulin production and increased hepatic glucose output. As insulin resistance is a cornerstone in developing type 2 diabetes, clinical approaches to managing this condition focus on improving peripheral tissue responsiveness to the action of insulin. Although skeletal muscle is the primary site of insulin-mediated glucose disposal, therapeutic strategies targeting skeletal muscle to improve insulin sensitivity have had limited success. Small organic molecules have been tested as potentiators of insulin receptor activation or inhibitors of protein tyrosine phosphatase 1B as approaches to increase insulin sensitivity in skeletal muscle. However, concerns about selectivity, the route of administration, and the risk of hypoglycemia have raised questions about the therapeutic effectiveness of these compounds.^{1,2} Nonetheless, given the central role of insulin resistance as an early factor in developing type 2 diabetes, improving insulin sensitivity in skeletal muscle remains an important clinical strategy.

Many antidiabetic agents have phytochemicals as lead structures. This includes metformin, a mainstay of type 2 diabetes treatment, which was inspired by a guanidine from Galega officinalis.³ Plants from the genus Artemisia, such as, A. annua (qinghaosu; the source of artemisinin) and Artemisia dracunculus (Russian tarragon) also have a long history of antidiabetic medicinal use.⁴ The importance of Artemisia species in providing phytochemicals for medicinal purposes is highlighted by the 2015 Nobel Prize for Medicine or Physiology being awarded to Youyou Tu for the discovery and development of artemisinin as an antimalarial drug.⁵ An ethanol extract from Artemisia dracunculus L. (Asteraceae), termed PMI-5011, reduces blood glucose levels and improves insulin levels in murine models of obesity-induced insulin resistance.⁶⁻⁸ PMI-5011 also enhances insulin signaling in murine and primary human skeletal muscle cells.^{7,9-11} Bioactivity-guided fractionation identified five compounds in PMI-5011 that affect glucose metabolism in vitro: (i) 4,5-di-O-caffeoylquinic acid, (ii) davidigenin, (iii) 6-demethoxycapillarisin, (iv) 4′-O-methyldavidigenin (1; 4,2′-dihydroxy-4′-methoxydihydrochalcone, syn.DMC-1) and its isomer (v) 4-O-methyldavidigenin (2; 2′,4′-dihydroxy-4-methoxydihydrochalcone, syn. DMC-2).¹²⁻¹⁶ Among them, purified 2 has demonstrated pronounced hypoglycemic effects by reducing blood glucose levels within six hours of administration in obese, insulin resistance mice.⁸ Hence, 2 is a designated bioactive marker of PMI-5011.

However, this designation remains accompanied by important questions that arise from the concept of bioactivity-guided fractionation, which by definition deconstructs the chemistry of an investigated extract. Being reductionist in nature, bioactivity-guided fractionation can fail to capture the therapeutic potential related to compound interactions in complex mixtures, such as PMI-5011. Backed by ethnobotanical knowledge, evidence is growing that herbal extracts exert their health effects holistically. Botanicals work as intact, complex chemical mixtures, in which multiple compounds canact on multiple pharmacological targets: a concept termed “polypharmacology”,^{17,18} Therefore, advancing the development of PMI-5011 and its bioactives as rationale therapeutic agents in treating insulin resistance requires an approach that decomposes A. dracunculus polypharmacology through selective modification rather than single-target decomposition of the crude extract.

To this end, the present study used the DESIGNER^{19,20} approach to Deplete and Enrich Select Ingredients to Generate Normalized Extract Resources as a means of determining more precisely, which compounds and their combinations within the extract have the desired biological activity. The new polypharmacological tool consisted of a knock-out extract (KOE) of PMI-5011, in which the previously identified bioactives, 2 and 1, were removed selectively. The biological properties of this KOE were then compared with those of unaltered PMI-5011, a mixture of primarily 2 and its regioisomer 1 termed methyldavidigenin knocked-out fraction (KOF), and synthetic 2. The objective of the present study was to determine the pharmacological contribution of the two methyldavidigenin derivatives to the overall effects of PMI-5011 on glucose homeostasis, both in vitro and in vivo. Located at the chemistry-biology interface, this study employed orthogonal phytochemical methodology and took a highly integrated in vitro/in vivo pharmacological approach to rationalize the therapeutic potential of a complex botanical extract in triggering diabetes resilience mechanisms.

Results and Discussion:

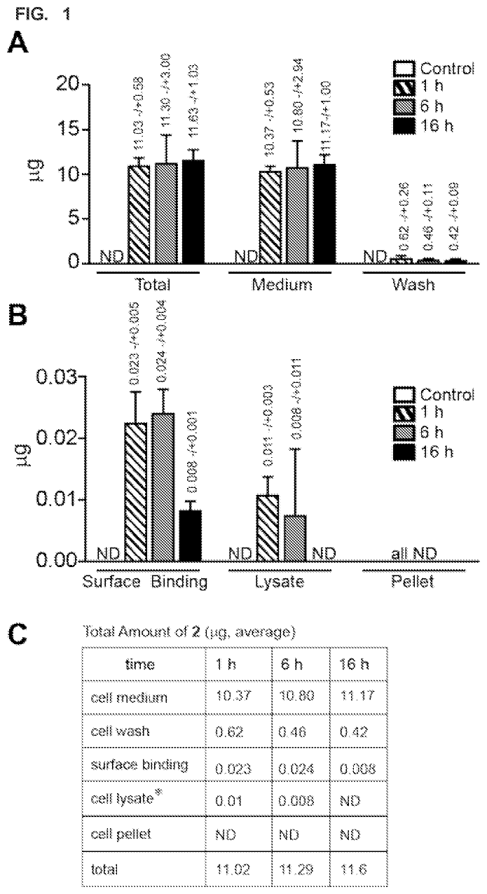

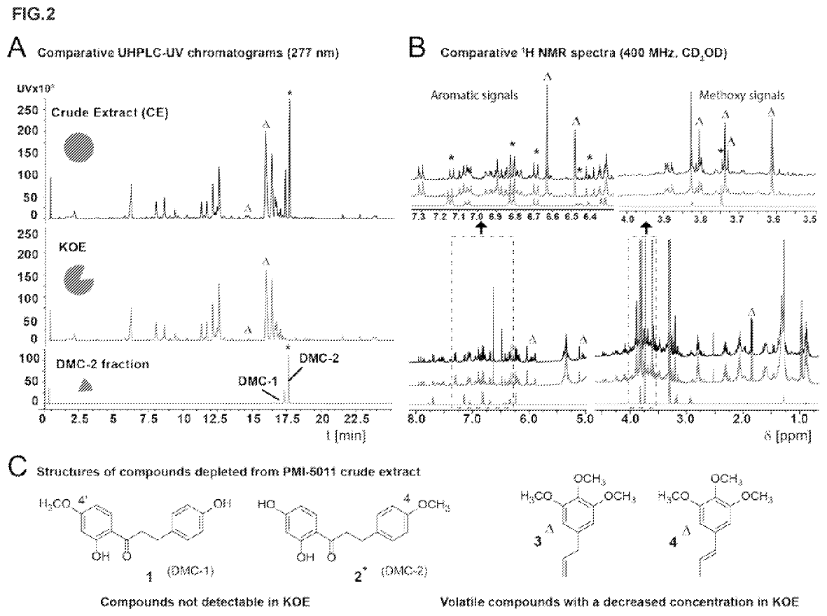

Designated Bioactive Principles in Russian Tarragon. A standardized ethanol extract of A. dracunculus, PMI-5011, reduced blood glucose levels and improved insulin sensitivity in mouse models of genetic and diet-induced obesity.^{7,21} Enhanced insulin signaling in murine and primary human skeletal muscle cells in vitro by PMI-5011 indicated that this extract affects glucose metabolism in skeletal muscle.^{9,11,14} Without wishing to be bound by theory, these findings indicated that bioactive small molecules from PMI-5011 can enter skeletal muscle cells. A kinetic study on myotube cells with PMI-5011 revealed that the bioactive chalcone, 2 (for structure see FIG. 2), binds to the cell surface within one hour, is internalized between one and six hours, but is undetectable after 16 hours (FIG. 1). Along with the quantification of 2 by LC-MS during the cell culture experiment, it should be noted that other designated bioactive compounds from PMI-5011, namely, sakuranetin, 6-demethoxycapillarisin, and davidigenin were also detected in the samples from treated cells. These compounds were not quantified due to the non-availability of reference standards for calibration. However, their presence approximately followed the changes observed in the concentrations of 2, with relatively large amounts in the cell culture medium and much smaller amounts bound to cell surfaces and present in the cell lysate of treated cells. Like 2, these compounds were not detected in the pellets of treated cells.

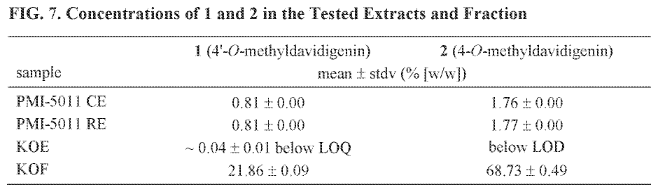

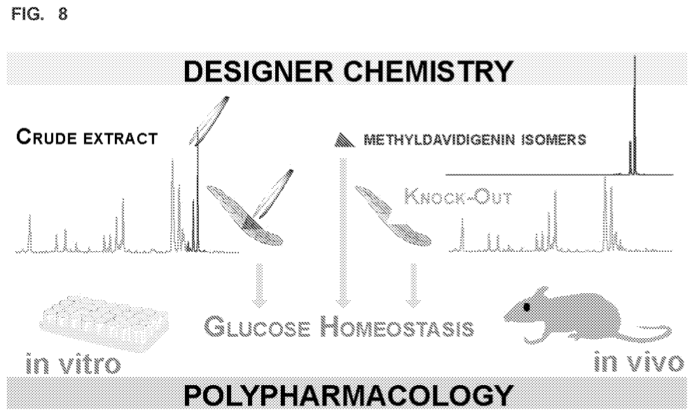

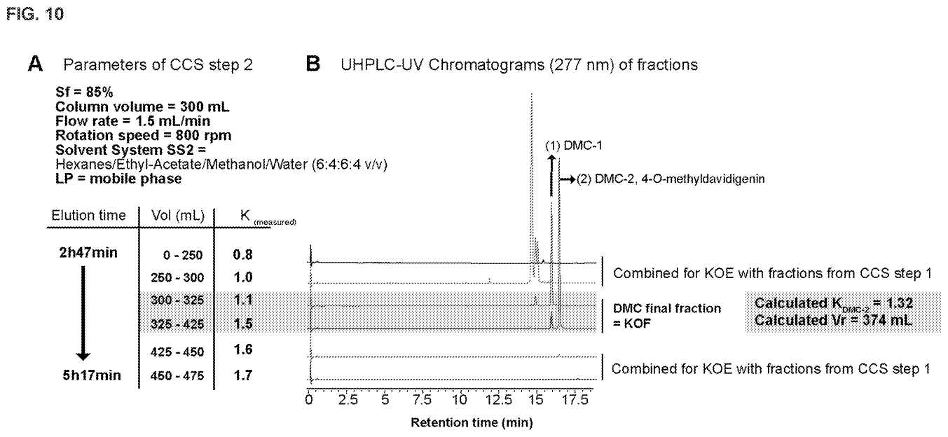

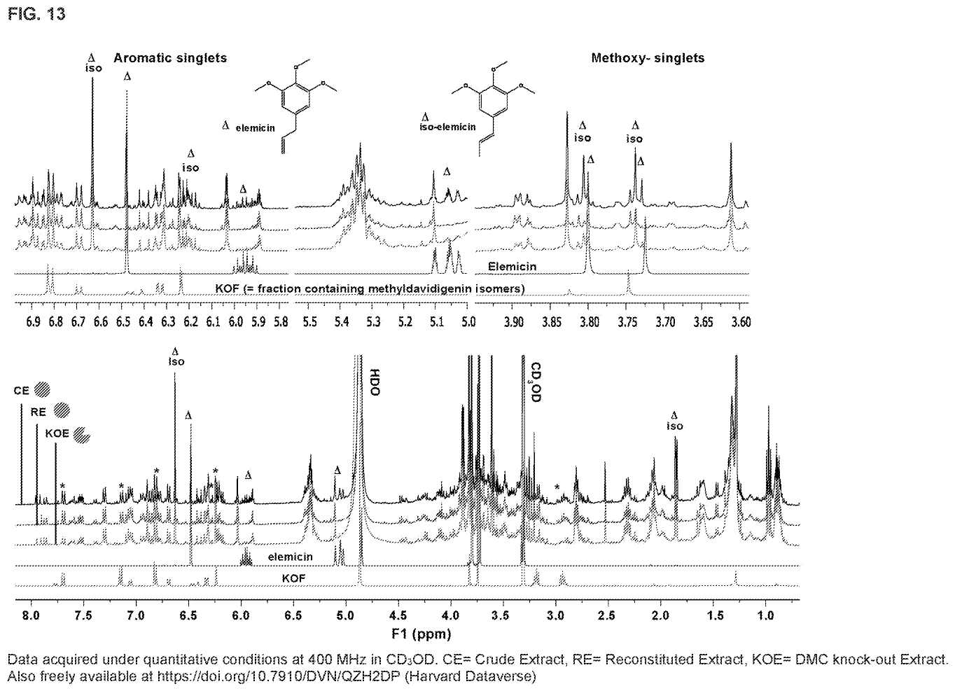

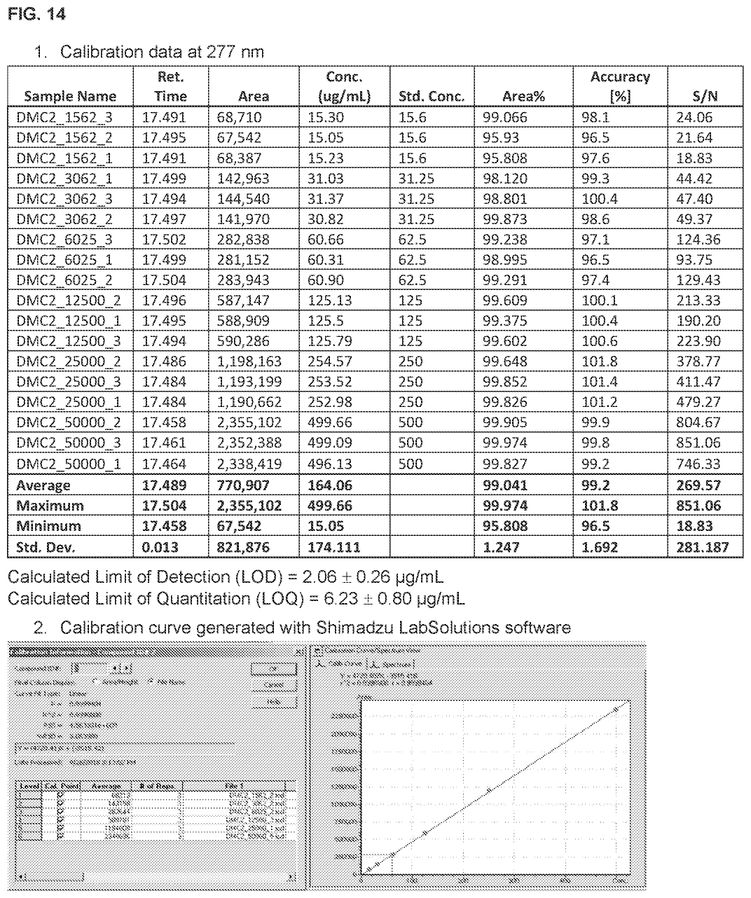

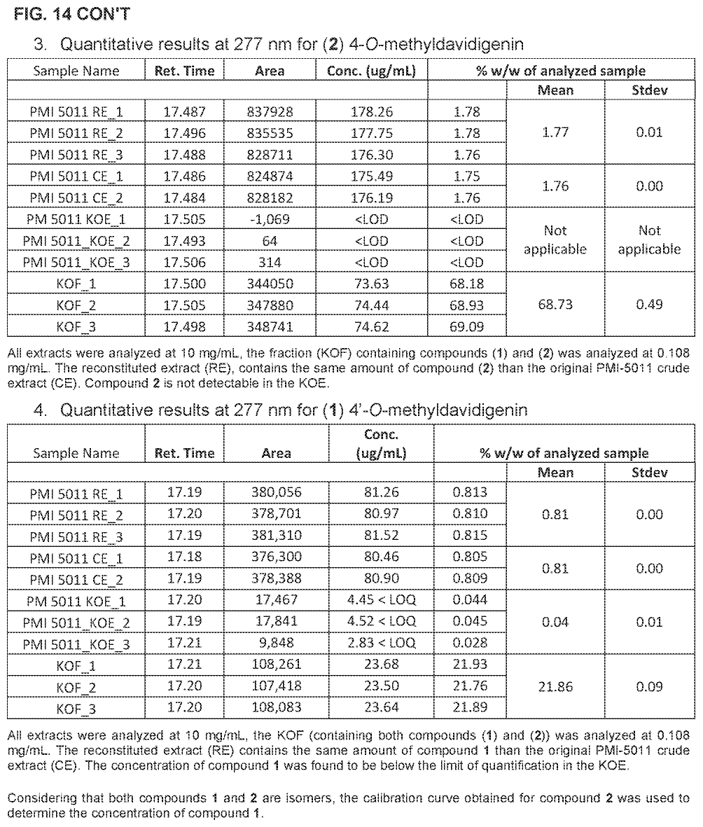

Preparation of Knock-Out Extracts (KOEs) as Pharmacological Tools. Two countercurrent separation steps (CCS, see also FIG. 10 and FIG. 11) transformed the original PMI-5011 into a knock-out extract (KOE), in which 1 and 2 were removed selectively (as per quantitative UHPLC-UV; FIG. 7). As 2 forms a critical pair of separation with its regioisomer, 1, both compounds were co-depleted from the original extract. The two CCS steps also led to the production of a methyldavidigenin knocked-out fraction (KOF) containing the two isomeric compounds 1 and 2. In order to evaluate the effect of the CCS process on the phytochemical integrity of PMI-5011 and its KOE derivative, a reconstituted extract was prepared by performing the full CCS followed by recombining all eluents including the KOE and the KOF. The reconstituted extract (RE) served as both a phytochemical and biological control. NMR and UHPLC metabolomic profiles documented the preservation of the chemical integrity of the KOE compared to PMI-5011, and evaluated the effectiveness of 1 and 2 depletion (FIG. 2, FIG. 13 and FIG. 14).

All extracts were analyzed at 10 mg/mL, the KOF was analyzed at 0.108 mg/mL. The reconstituted PMI-5011 extract (RE) contained the same amount of 1 and 2 as the original PMI-5011 crude extract (CE). LOD: Limit of detection for 1 and 2 (2.06±0.26 μg/mL), LOQ: limit of quantification (6.23±0.80 μg/mL).

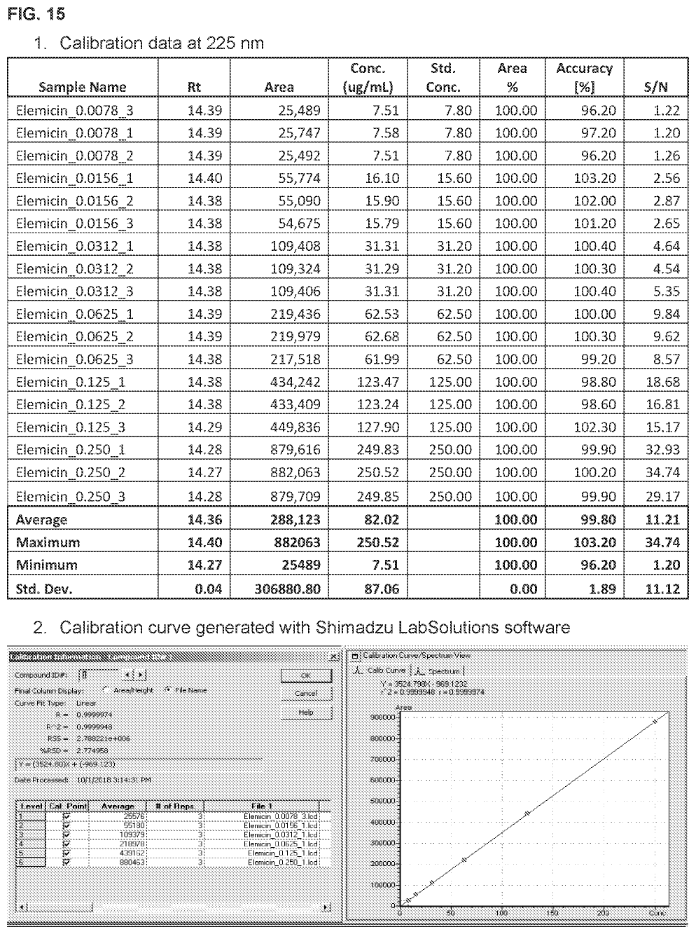

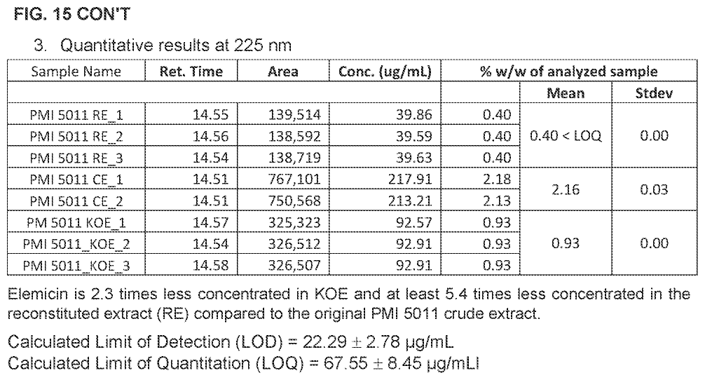

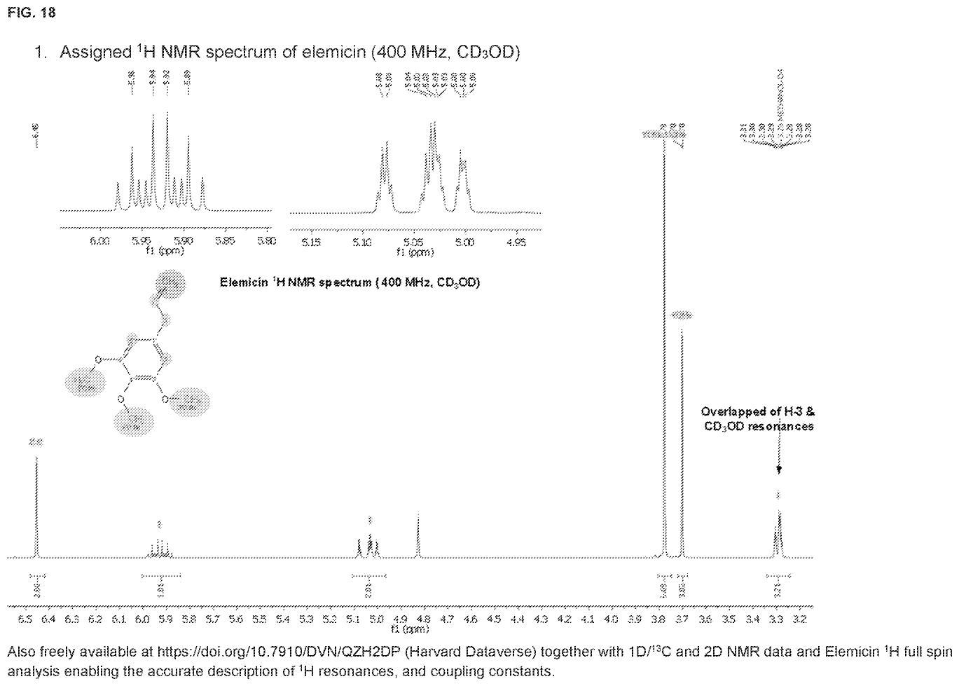

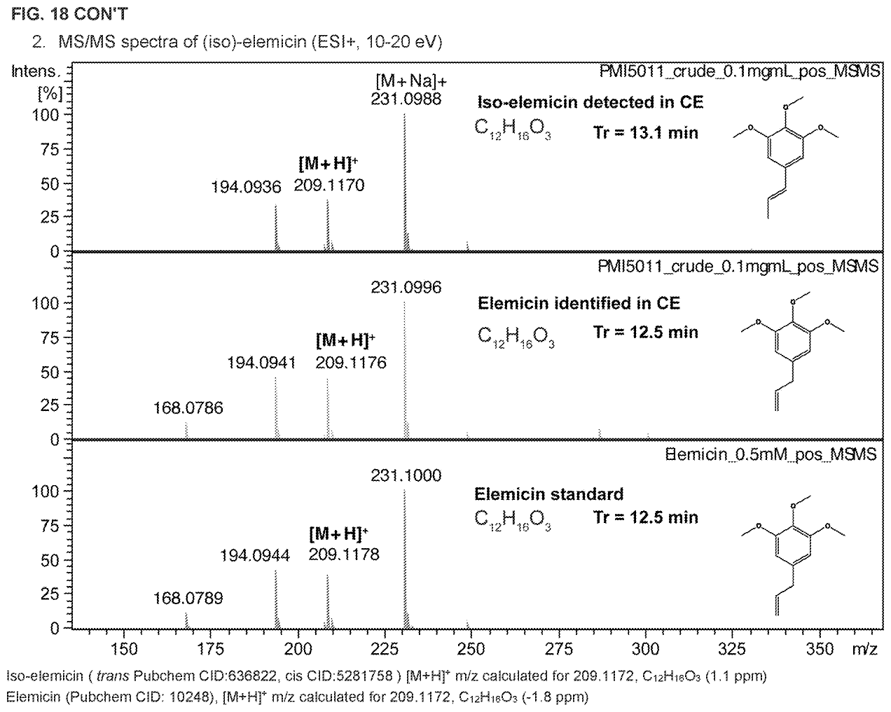

Comparison of all UHPLC-UV and 1H NMR fingerprints (FIG. 2) showed that the KOE retained all phytochemical features of the original PMI-5011, with the exception of two volatile isomeric phytoconstituents, namely, elemicin (3) and iso-elemicin (4). Both compounds, identified herein by NMR and MS analyses (FIG. 2, FIG. 13, FIG. 14, FIG. 18), are reported to be abundant methoxy-allylbenzenes in Russian tarragon and, thus, PMI-5011.4 Their concentrations were decreased in both KOE and PMI-5011 RE (FIG. 15), as a result of the inevitable solvent evaporation for the production of dried extract necessary for conducting bioassays. In a similar manner, the multiple steps of solvent evaporation and sample reconstitution during any bioactivity-guided fractionation process, would lead to a significant decrease of both 3 and 4 concentrations in the produced fractions, thereby hampering their detection and isolation as potential bioactive compounds. In fact, data pertaining to ¾ bioactivities are scarce, despite its relative abundance in aromatic plants.^{22,23}

Complemented by the phytochemical methods, the in vivo bioactivity profiles obtained originally with unaltered PMI-5011 were compared to those obtained with KOE, the KOF, synthetic 2, and the RE. Tested at the same concentration, a KOE that will demonstrate a loss of activity compared to the original PMI-5011, together with a recovery of the activity with the KOF will, therefore, confirm that 2 and its regioisomer, 1, are responsible for the measured effect.

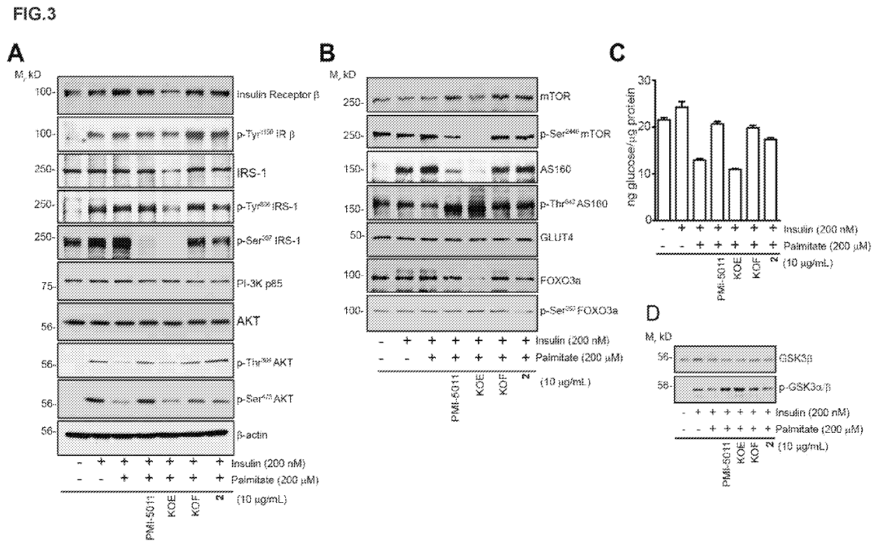

Insulin Signaling. Insulin signaling in palmitate-treated insulin-resistant C2C12 myotubes was assayed after treatment with the PMI-5011, the KOE, the KOF, and the synthetic 2 (FIG. 3), each at 10 μg/mL. PMI-5011 mediated upregulation of insulin-dependent AKT phosphorylation at threonine 308 and serine 273 in the presence of palmitate was lost with the KOE, but restored with the KOF and purified 2. Depletion of 1 and 2 was also associated with reduced steady-state levels of IRO, IRS-1, mTOR, and FOXO3a, along with further decreased AS160 levels compared to PMI-5011. In addition, tyrosine phosphorylation of IRS-1 and serine phosphorylation of mTOR were diminished with the KOE.

In contrast, KOE treatment still supported PMI-5011-mediated inhibition of IRS-1 serine phosphorylation as well as increased AS160 phosphorylation, just as observed with unaltered PMI-5011 (FIG. 3). This indicated that compounds other than 1 and 2 are indeed responsible for these activities. While increased glycogen content resulting from PMI-5011 treatment was absent with the KOE, KOF or synthetic 2 restored this activity (FIG. 3). This correlated with the increased GSK3 phosphorylation observed with the KOE compared to synthetic 2 (FIG. 3).

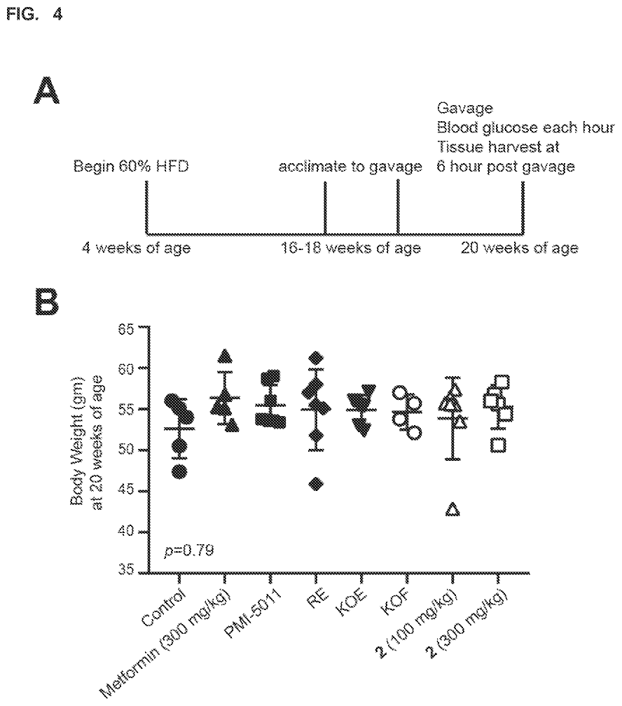

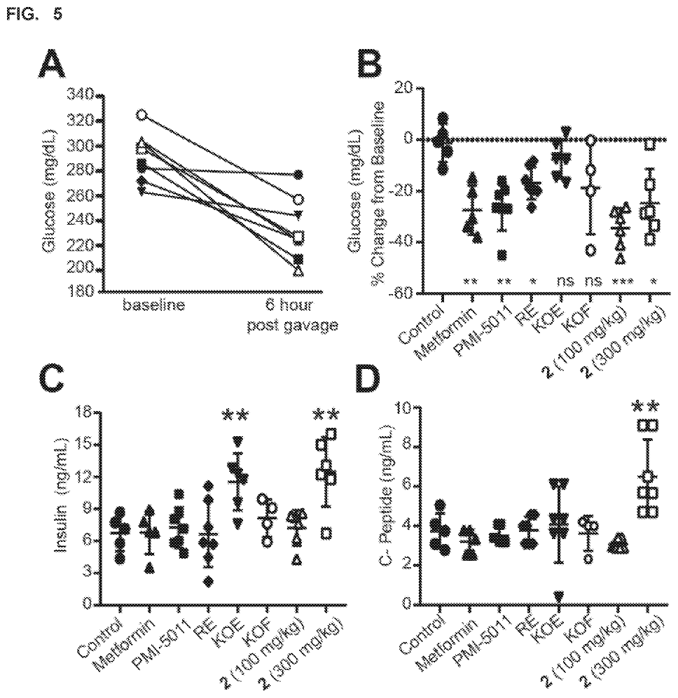

Glucose Homeostasis. The next major question was, whether the effect of PMI-5011 on glucose homeostasis in vivo could correlate with the levels and activities of 2. To address this, C57BL/6J male mice were fed a very high fat diet (60% kcal fat, VHFD) over 16 weeks to induce obesity, prior to testing the ability of PMI-5011 (500 mg/kg), KOE (500 mg/kg), KOF (100 mg/kg), and purified 2 at 100 or 300 mg/kg body weight to alter blood glucose levels (FIG. 4). Metformin (300 mg/kg) was used as a positive control. The mice were assigned randomly to each treatment group, and there was no difference in body weight among them (FIG. 4). As shown in FIG. 5, a 6 hour treatment with metformin, PMI-5011, PMI-5011-RE, and synthetic 2 significantly reduced blood glucose levels in the obese male mice. Blood glucose levels were not changed by the KOE, and were also unchanged by the KOF, for example, due to the variability and low number of animals (N) related to the dose and limited sample.

Unexpectedly, treatment with KOE and the higher dose of the synthetic 2 increased insulin levels significantly (FIG. 5). This was accompanied by increased C-peptide levels resulting from treatment with synthetic 2, but not with the KOE (FIG. 5). Interestingly, despite the inevitable decrease in 3 and 4 concentrations, the in vivo activities of the RE were statistically equivalent to those of the original PMI-5011. This indicated that these two major volatile constituents do not contribute to the effects of PMI-5011 on blood glucose and insulin levels.

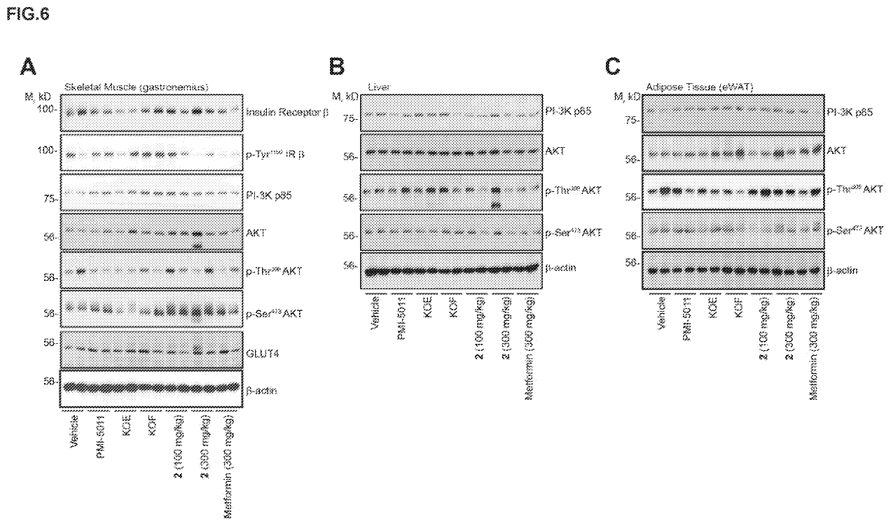

To determine if the change in blood glucose levels corresponded to an effect on insulin responsiveness in skeletal muscle, western blot analysis was used to interrogate insulin signaling in mixed gastrocnemius. As found in the C2C12 myotubes (FIG. 3), steady-state levels of IRβ decreased with KOE treatment, but were maintained by KOF (FIG. 6). IRO tyrosine1150 phosphorylation was enhanced by the KOF, but not by synthetic 2, independent of concentration, indicating the involvement of 1 in this activity. AKT levels were not altered substantially by the treatments, but there was an upregulation of AKT serine273 phosphorylation with PMI-5011 that was absent with KOE treatment, restored with the KOF, and more pronounced with pure 2. Compared to the vehicle control, AKT threonine308 phosphorylation was not enhanced by PMI-5011, KOE, or KOF (FIG. 6). Unlike their in vitro effect (FIG. 3), KOF and pure 2 in vivo at 100 mg/kg increased levels of the PI3K-p85 subunit in skeletal muscle within 6 h (FIG. 6). To determine if the changes in insulin signaling were specific to skeletal muscle, PI-3K p85, AKT, threonine308 and serine273 phosphorylation in the liver and gonadal adipose tissue were assayed. In the liver, a clear pattern of changes in the PI-3K p85 or AKT protein levels did not emerge. Regulation of AKT serine273 phosphorylation by KOE and KOF was less robust in the liver, but unlike skeletal muscle, AKT threonine308 phosphorylation increased with PMI-5011, treatment (FIG. 6). AKT serine273 phosphorylation was not regulated by PMI-5011, KOE, or KOF in adipose tissue. AKT threonine308 phosphorylation was substantially reduced by PMI-5011, KOE, and KOF, but not by treatment with the synthetic 2. An upregulation in total AKT also occurred in adipose tissue when treating with the KOF or 2 at 300 mg/kg body weight.

Elucidation of Complex Mixtures with Complex Modes of Action. A number of pre-clinical studies have reported that a complex mixture of compounds found in the PMI-5011 ethanol extract of A. dracunculus improves glucose metabolism and insulin responsiveness in obesity-induced insulin resistance.^{6,7,9-11} However, the lack of a more detailed understanding of how the complex mixture of phytochemicals in PMI-5011 interacts to improve insulin sensitivity has hampered the development of PMI-5011 as a treatment of insulin resistance in type 2 diabetes. This study used the DESIGNER approach to elucidate the impact of individual compounds present in PMI-5011 on skeletal muscle insulin signaling pathways critical to maintaining insulin sensitivity. Notably, this is the first study to demonstrate that a DESIGNER-generated KOE can be produced in sufficient quantities to dissect in vivo the physiological impact of an individual compound present in a complex mixture of phytochemicals.

While the classical bioactivity-guided fractionation approach is reductionistic and aimed at pairing bioactivity with one or few isolated compounds, the DESIGNER approach enables new correlations by connecting the bioactivity of target compounds with their presence and absence and concentration in complex mixtures, such as in PMI-5011, but also KOF, as exemplified in the following. Both KOF and synthetic 2 had to be dosed ˜40 and ˜60 times higher, respectively, than the corresponding concentration of 2 in the crude extract, in order to obtain a biological effect equivalent to the effect measured with the crude PMI-5011. In fact, when testing the crude PMI-5011 at 10 μg/mL, the effective concentration of 2 is only 0.17 μg/mL (FIG. 7), which is 60-fold less than purified 2 tested at 10 μg/mL. This indicates that the effects of 2 on glucose homeostasis are non-specific. However, when comparing the activities between the crude PMI-5011 and KOE, the specific loss of activities could still be pinpointed to the depletion of the methyldavidigenins. Collectively, these results indicate that methyldavidigenins are much more active within the PMI-5011 matrix than as pure compounds. Also, other PMI-5011 constituents may promote the bioavailability or PK behavior of methyldavidigenins so they can exert their biological effect at lower concentrations. Similarly, testing KOF at 100 mg/kg led to an effective concentration of 68.7 mg/kg of 2 and 21.8 mg/kg of 1, when considering the methyldavidigenin concentrations in this fraction (FIG. 7). Hence, the isomeric methyldavidigenin mixture displayed identical bioactivities compared to purified 2 at 100 mg/kg, thereby indicating a combinatorial effect of these regioisomers on the measured bioactivities. Further studies will uncover the effect of other PMI-5011 constituents on 2 and 1 bioavailability, and to determine whether their effects on glucose homeostasis are synergistic, additive, or related by other mechanisms.

Evaluation of the polypharmacological properties of KOE confirmed that the selectively removed 2 and 1 contribute to certain, but certainly not all, biological activities of the whole extract. The two-step CCS-based production scheme removed 2 and 1 and preserved equivalence to the original PMI-5011 by maintaining the proportionality between the multiple phytoconstituents (metabolomic profile), except for the labile/volatile methoxy-allylbenzenes, 3 and 4. However, results obtained in this study indicate that these constituents do not contribute to the effects of PMI-5011 on blood glucose and insulin levels.

The outcomes demonstrate that 2 (and likely 1) is the major bioactive principle in PMI-5011 that enhances AKT activation in vitro (FIG. 3) as well as in vivo in skeletal muscle with insulin resistance (FIG. 6). In vivo AKT activation occurred in skeletal muscle when acutely exposed to PMI-5011 or KOF, but was not observed with KOE, and did not occur in adipose tissue or the liver. Interestingly, the insulin levels were increased by both KOE and pure 2 at 300 mg/kg within 6 h. The higher dose of 2 at 300 mg/kg corresponded to increased C-peptide levels were indicative of stimulated insulin secretion. Hence, 2 at high concentration had an additional impact on pancreatic function in obesity-induced insulin resistance that was absent at lower levels of this compound, and/or when 2 acts in combination with other phytochemicals present in the unaltered PMI-5011 extract. Conversely, C-peptide levels were unchanged by KOE, where the increased insulin levels with KOE reflect a modulating impact of methyldavidigenins on hepatic and extrahepatic insulin clearance.²⁴⁻²⁶

All of these findings correlate with changes in the effect of PMI-5011 on AKT phosphorylation in skeletal muscle. This primarily occurs in skeletal muscle within 6 h, but may not be entirely specific to the skeletal muscle, as increased AKT phosphorylation at threonine308 occurred in the liver as well. Although adipose tissue is important in determining glucose homeostasis and insulin sensitivity in obesity, the glucose-lowering effect of PMI-5011 and KOF did not involve enhanced insulin signaling in the visceral adipose tissue.

The DESIGNER extract approach also revealed that methyldavidigenins possess previously unrecognized mechanisms of action within PMI-5011. The steady-state levels of several proteins are differentially regulated by 2 and KOE, indicating the balance between protein synthesis and degradation is influenced by 2. Earlier studies showed that PMI-5011 inhibits proteasome activity.²⁷ Reduced levels of IRS-1, AS160, or mTOR upon KOE treatment indicate that methyldavidigenins are responsible for the effect of PMI-5011 on protein steady-state levels. However, treatment by both PMI-5011 and KOE affected the phosphorylation of IRS-1 almost equally in the C2C12 skeletal muscle cells. This indicates that other phytochemicals in PMI-5011 are responsible for this anti-inflammatory effect, which is in line with the polypharmacological paradigm of botanicals. Considering the relatively high amount of 3 and 4 in PMI-5011 (see FIG. 15), we will validate its biological contribution to any polypharmacological effects of PMI-5011.

Concluding Remarks. In summary, this study exemplifies the use of the DESIGNER approach, with the production of a KOE as a polypharmacological tool for the decryption of biological activities of compounds within a complex mixture such as PMI-5011. The KOE helped decipher the contribution of 2 and its regioisomer 1 on glucose homeostasis. The outcomes confirm that 2 is a major bioactive principle that enhances AKT activation in vitro and in vivo in skeletal muscle in the presence of insulin resistance. In vivo AKT activation occurs in skeletal muscle when acutely exposed to PMI-5011 or the KOF, but is not observed with the KOE, and does not occur in adipose tissue or the liver. The DESIGNER approach also enhanced our understanding of the role of the designated bioactives to the overall in vivo effects of PMI-5011. The demonstrated multiple biological effects of 2 and its regioisomer, 1, on glucose homeostasis support the development of standardized PMI-5011 for the complementary treatment of type-2 diabetes.

Experimental Section:

Plant Material. Artemisia dracunculus was grown in a Rutgers University greenhouse facility in New Brunswick, N.J. (40° 28′41.9″N 74° 26′15.7″W) using commercially available seed obtained from Sheffield's Seed Company, Locke, N.Y., USA. Voucher specimens are retained at Rutgers University Chrysler Herbarium (CHRB) under the guidance of a taxonomy specialist.²⁸

Extraction and Isolation. The preparation of the PMI-5011 botanical extract from Artemisia dracunculus and detailed information about its quality control and biochemical characterization have been reported previously.^{6,8,12,13,29,30} Earlier studies led to the isolation of five compounds with in vitro activity.¹² Compound 2 was also synthesized as previously described.⁸

Preparation of the Knock-Out Extract (KOE) and the Knocked-Out Fraction (KOF). The depletion of 2 from PMI-5011 was achieved by two successive steps of countercurrent separation (CCS) using orthogonal biphasic solvent systems (SSs) composed of hexanes, ethyl acetate, ethanol, and water (5:4:4:4, v/v) as SS1, and hexanes, ethyl acetate, methanol, and water (6:4:6:4, v/v) as SS2 (see FIG. 10 and FIG. 11). The partition coefficients (K; a value expressing the ratio of the concentration of the compound between the upper and lower phase of the two-phase solvent system) of 2 were 4.53 in SS1 and 1.32 in SS2. The first CCS step used a hydrostatic Spot-Prep SCPC-250-B (Armen Instrument SAS, Gilson, Inc.), equipped with a 250 mL column, which was operated in reversed-phase mode using SS1 (25 mL/min at 2800 rpm). Under these conditions, the stationary phase fraction (Sf) at equilibrium was 0.52. For injection, 1.306 g of PMI-5011 was dissolved in 7.5 mL of each upper and lower phase. A fraction enriched with (2) eluted from K=3.30 (550 mL) to 5.80 (875 mL). The rest of PMI-5011 was recovered fully by re-combining the liquid stationary and all mobile phase that eluted before and after 2.

To generate the KOE, the fraction enriched in 2 (42 mg) underwent a second CCS step using a hydrodynamic High-Speed Countercurrent Chromatography (HSCCC) apparatus (TBE-300B, Tauto Biotech Co., Shanghai, People's Republic of China), equipped with a 300 mL column, operated in reversed-phase mode with SS2 (1.5 mL/min at 800 rpm; Sf=0.85). Even in this orthogonal system, 2 eluting at 300 (K=1.0, 3 h 20 min) to 425 mL (K=1.5) post injection, showed co-elution with its regioisomer, 1. Collectively, the two-step process yielded 1.122 g of KOE and 25.3 mg of the KOF that contained all of 2 and 1 (FIG. 2). While the mass recovery of the initial PMI-5011 was 87.8% (see FIG. 11), the observed loss was not compound specific and only due to the sample filtration and handling during injection (FIG. 10-FIG. 12, provide the analytical details). As a control, a second batch of PMI-5011 (1.274 g) underwent the same two CCS steps, but all fractions including those containing compounds 1 and 2 were recombined.

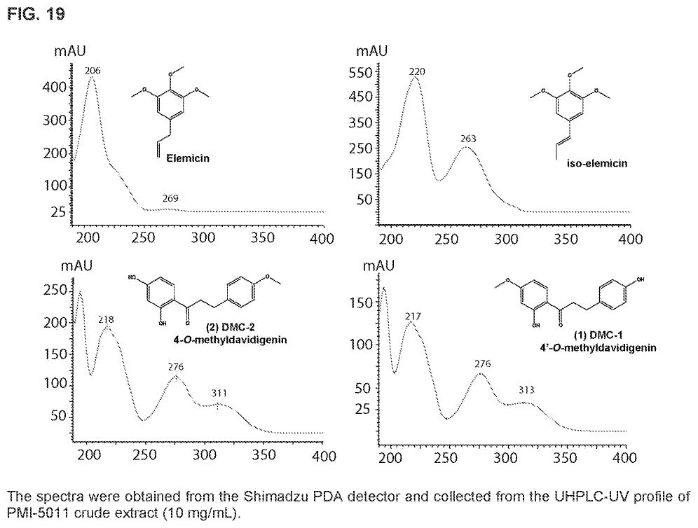

Comparative UHPLC-UV Analyses and Quantitation of 2. UHPLC analyses utilized a Shimadzu UHPLC equipped with a Kinetex XB-C18 column (2.1×50 mm, 1.7 μm, part #00B-4498-AN, Phenomenex) and a Diode Array Detector (DAD, Shimadzu SPD-M20-A). The elution gradient (0.8 mL/min) was composed of (A) water and (B) acetonitrile, both with 0.1% formic acid, as follows: 5% B from 0 to 2 min, going to 25% B at 12 min and during an additional 3 min, then reaching 70% B by 21 min and maintaining for an additional 3 min. Extracts were dissolved at 10 mg/mL, KOF at 0.10 mg/mL, in 70% HPLC-grade acetonitrile. Under these conditions, the retention times (tR) of 1 and 2, were 17.20 and 17.50 min, respectively. The elemicins 3 and 4 co-eluted at 14.50 min. For quantification (see FIG. 13, FIG. 14, and FIG. 19), 2 and 3/4 calibration curves were analyzed at 277 and 225 nm, respectively.

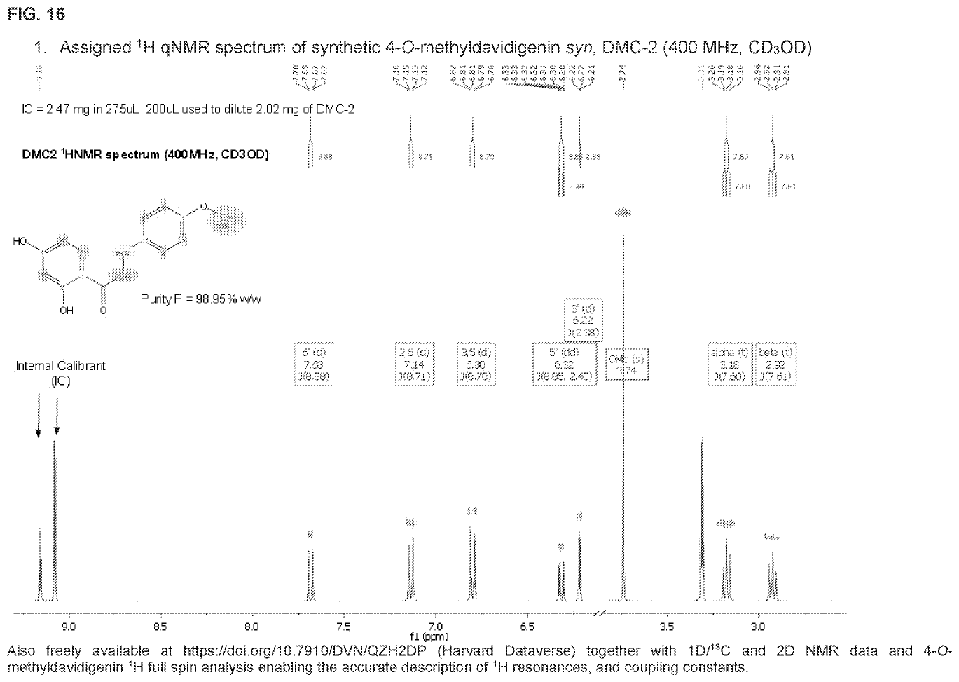

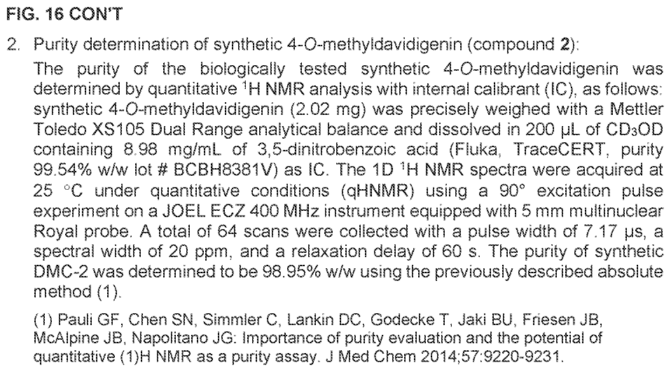

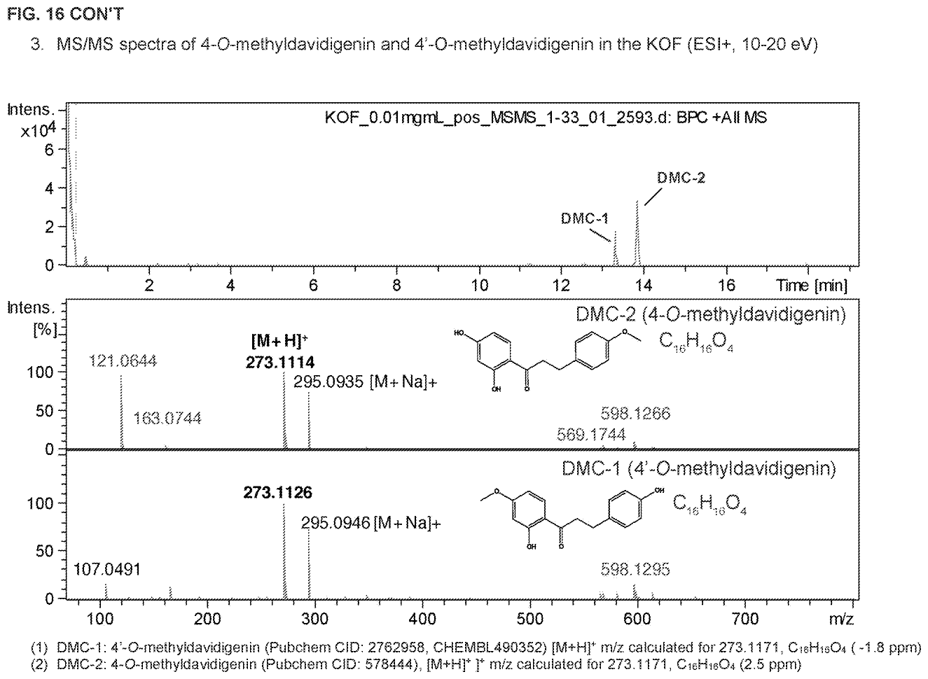

Comparative ¹H NMR Fingerprints. For NMR analysis, exactly weighed (±0.01 mg) samples of the original crude extract and KOE (8-10 mg) as well as the KOF (ca. 1.5 mg) were dissolved in 300.0 μL CD3OD (99.8% D; Cambridge Isotope Laboratories Inc., Andover, Mass., USA). Using calibrated glass pipets, 200.0 μL of each solution were transferred into 3 mm NMR tubes (S-3-HT-7, Norell Inc., Landisville, N.J., USA). The ¹D ¹H NMR spectra were acquired at 25° C. under quantitative conditions (qHNMR) using a 90° excitation pulse experiment on a JEOL ECZ 400 MHz instrument equipped with 5 mm multinuclear Royal probe. The probe frequency was automatically tuned and impedance matched before each acquisition. Off-line data analyses were performed using Mnova software (v.11.0.3, MestreLab Research S.L., A Coruna, Spain). The Supporting Information (FIG. 16) provides the purity determination of synthetic 2.

Animal Experiments. Male C57BL/6J mice were obtained from Jackson Laboratories (Bar Harbor, Me.). All animal experiments were approved by the Rutgers University Animal Care and Use Committee (Protocol #04-023). The animals were housed singly with a 12 h light-dark cycle at 24° C. At four weeks of age, the mice were placed on a very high fat diet (VHFD, 60 kcal % fat, D12492, Research Diets) for 16 weeks. Body weight was measured weekly. The mice were acclimated to gavage over a 2-week period prior to administering the experimental materials, vehicle control (Labrasol), or metformin via gavage. Blood glucose levels were assayed at baseline and each hour after administering the extracts. The mice were euthanized and tissue collected for analysis 6 h after gavage.

Cell Culture. Murine C2C12 (ATCC; #CRL-1771) or rat L6 myoblasts (ATCC, #CRL-1458) were cultured in DMEM, high glucose (25 mM) with 10% FBS, 2 mM glutamine, and antibiotics (100 units/mL penicillin G and 100 μg/mL streptomycin) in a humidified chamber at 37° C. and 5% CO₂. To obtain fully differentiated myotubes, the medium was exchanged for DMEM, high glucose with 2% horse serum, glutamine, and antibiotics when the myoblasts reached confluence. The medium was replaced every 48 hours, and the cells were maintained in this medium until fully differentiated, when the medium was exchanged for DMEM, low glucose (5 mM) with 2% horse serum. The myotubes were fully formed by the fourth day post-induction. The experiments analyzing the impact of each experimental material on insulin signaling in the presence of palmitate-induced insulin resistance were performed as described previously.³¹ To determine uptake of bioactives into the myotubes, the cells were treated with PMI-5011 (10 μg/mL) for 0, 1, or 16 h prior to medium collection. At each time point, the cells were thoroughly rinsed with D-Hanks, pH 7.4 at 4° C., followed by incubation with D-Hanks buffer, pH 4.0 at 4° C. for 30 min with gentle rocking to remove surface-bound compounds. Cells were then collected in denaturing buffer containing 50 mM Tris-Cl, pH 7.4 with 150 mM NaCl, 1 mM EDTA, 1% Igepal, 0.5% Na-deoxycholate, 0.1% SDS, protease inhibitors (1 μM PMSF, 10 μg/mL aprotinin, 1 μg/mL pepstatin, 5 μg/mL leupeptin). The harvested cells were sonicated on ice and the supernatant and pellet were collected by centrifugation at 13,400×g for 10 min at 4° C.

UPLC/MS Analysis of Cell Preparations. Each sample of cell culture medium (or portion thereof) or cell culture preparation was partitioned in triplicate with an equal volume of ethyl acetate, dried by rotary and subsequent SpeedVac evaporation, and resuspended in 250 μL 90% ethanol. The pellet samples were extracted directly with 90% ethanol. UPLC/MS analysis utilized a Dionex® UltiMate 3000 RSLC ultra-high-pressure liquid chromatography system, a photodiode array detector DAD-3000RS, and a Q Exactive Plus Orbitrap high-resolution high-mass-accuracy mass spectrometer (MS). Mass detection with an electrospray (ESI) interface was full MS scan with low energy collision induced dissociation (CID) from m/z 100 to 1000 in either positive or negative ionization mode. Substances were separated on a Phenomenex™ Kinetex C8 reverse d-phase column (100×2 mm, 2.6 μm/100 Å particles). The mobile phase consisted of two components: solvent A (0.5% ACS grade acetic acid in LCMS grade water, pH 3-3.5), and solvent B (100% LC-MS grade acetonitrile). The mobile phase gradient consisted of 95% A and 5% B to 5% A and 95% B over 30 min at 0.20 mL/min. Quantification used a calibration curve of 2 to convert the sample starting volumes into total amounts of 2, except for the pellets where nothing was detected. Identification of the other bioactive compounds utilized an in-house library of spectral data and database searches (reaxys.com, Elsevier RELX Intellectual Properties SA; SciFinder, American Chemical Society).

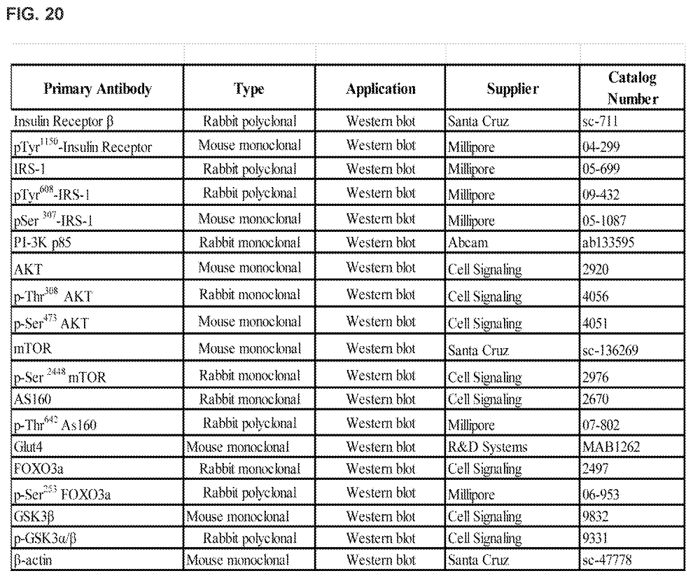

Protein Expression Analysis. Skeletal muscle, liver, and gonadal adipose tissue lysates were prepared from powdered tissue by homogenization in 25 mM HEPES, pH 7.4, 1% Igepal CA630, 137 mM NaCl, 1 mM PMSF, 10 μg/mL aprotinin, 1 μg/mL pepstatin, and 5 μg/mL leupeptin, and centrifugation at 14,000×g for 10 min at 4° C. Protein concentrations were determined using BCA assays (Thermo Fisher Scientific, Rockford, Ill.). The tissue supernatants (50 μg) were resolved by SDS-PAGE and subjected to immunoblotting using chemiluminescence detection (Thermo Fisher Scientific, Rockford, Ill.) and quantified as described.³² Nitrocellulose membranes were incubated with antibodies (FIG. 20) for 1-2 h at RT or overnight at 4° C.

Blood and Tissue Chemistry. Fasting glucose levels were measured in whole blood using a Breeze2 glucometer (Bayer, Leverkusen, Germany). Murine fasting insulin and C-peptide levels were assayed via ELISA (Crystal Chem, Downers Grove, Ill., USA). Glycogen content was assayed as directed by the manufacturer (Sigma).

Statistical Analysis. Distribution of body weight, blood glucose, insulin and C-Peptide levels were assessed using the D'Agostino-Pearson omnibus normality test. Statistical significance was determined using an unpaired two-tailed t test of the mean and standard deviation. GraphPad Prism 5 software was used for statistical analysis.

Safety Statement. No unexpected or unusually high safety hazards were encountered.

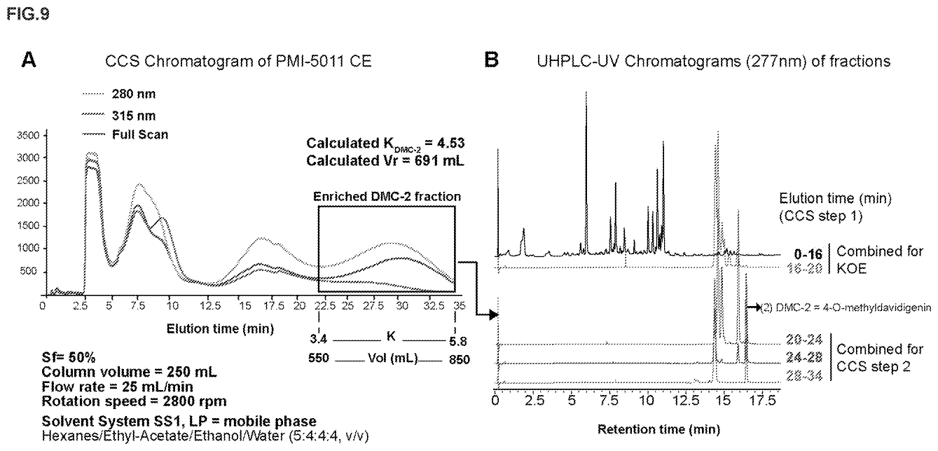

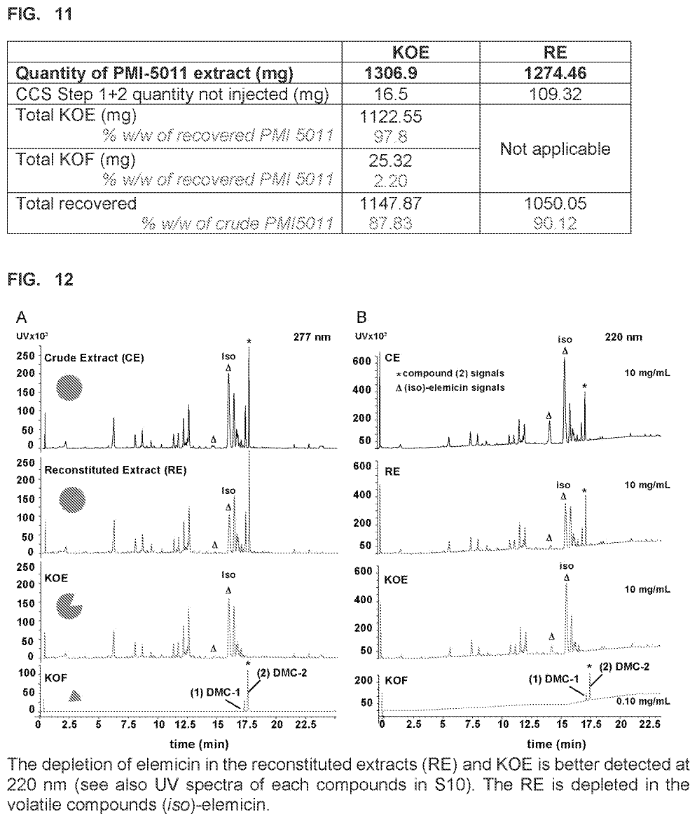

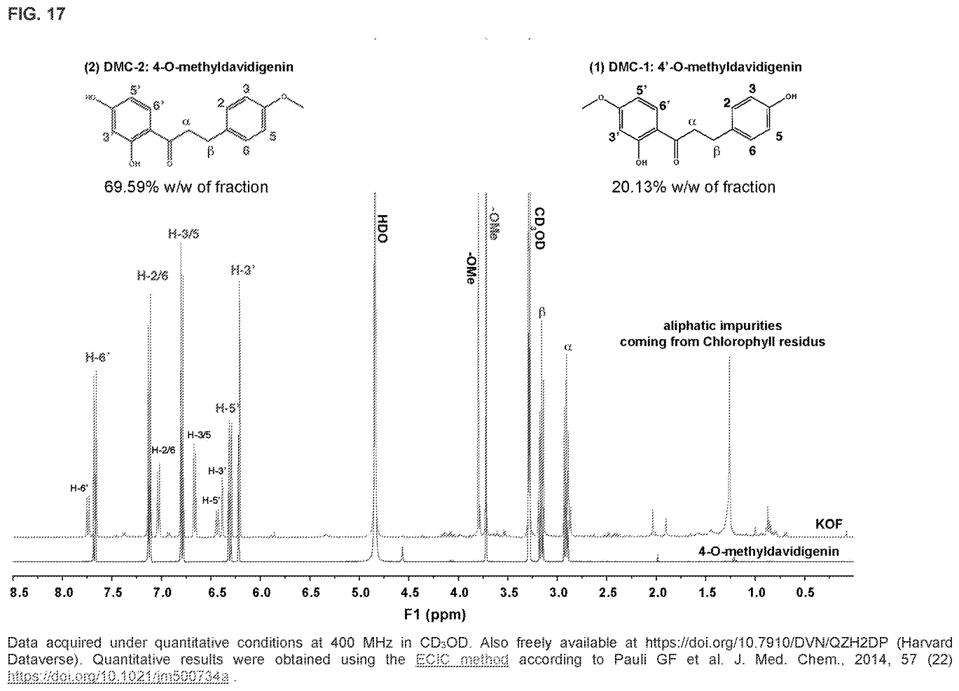

Supporting Information. Included are a note on the nomenclature of A. dracunculus PMI-5011 extract; the chromatograms illustrating the different CCS steps for the production of the KOE (FIG. 9 and FIGS. 10 S2 and S3, Supporting Information); the comparative UHPLC-UV and 1H NMR fingerprints of the different PMI-5011 extracts and KOF (FIG. 12 and FIG. 13); the quantitative data for 1 and 2, (FIG. 14), and for 3/4 (FIG. 15) in the different PMI-5011 extracts; the annotated NMR and MS/MS spectra of 1 and 2, (FIG. 16 and FIG. 17) and 3/4 (FIG. 18), as well as their UV spectra (FIG. 19). Additional information on the name, type, application, supplier and catalog number of all antibodies used in western blot analysis is also provided (FIG. 20).

### REFERENCES CITED IN THIS EXAMPLE

(1) Bailey, C. J.; Tahrani, A. A.; Barnett, A. H. Lancet Diabetes Endocrinol. 2016, 4, 350-359.
(2) Tahrani, A. A.; Barnett, A. H.; Bailey, C. J. Nature Rev. Endocrinol. 2016, 12, 566.
(3) Witters, L. A. J. Clin. Invest. 2001, 108, 1105-1107.
(4) Obolskiy, D.; Pischel, I.; Feistel, B.; Glotov, N.; Heinrich, M. J. Agric. Food Chem. 2011, 59, 11367-11384.
(5) White, N. J.; Hien, T. T.; Nosten, F. H. Trends Parasitol. 2015, 31, 607-610.
(6) Ribnicky, D. M.; Poulev, A.; Watford, M.; Cefalu, W. T.; Raskin, I. Phytomedicine 2006, 13, 550-557.
(7) Wang, Z. Q.; Ribnicky, D.; Zhang, X. H.; Zuberi, A.; Raskin, I.; Yu, Y.; Cefalu, W. T. J Nutr. Biochem. 2011, 22, 71-78.
(8) Ribnicky, D. M.; Kuhn, P.; Poulev, A.; Logendra, S.; Zuberi, A.; Cefalu, W. T.; Raskin, I. Int. J. Pharm. 2009, 370, 87-92.
(9) Kheterpal, I.; Coleman, L.; Ku, G.; Wang, Z. Q.; Ribnicky, D.; Cefalu, W. T. Phytother. Res. 2010, 24, 1278-1284.
(10) Obanda, D. N.; Cefalu, W. T. J. Nutr. Biochem. 2013, 24, 1529-1537.
(11) Wang, Z. Q.; Ribnicky, D.; Zhang, X. H.; Raskin, I.; Yu, Y.; Cefalu, W. T. Metabolism 2008, 57, S58-64.
(12) Logendra, S.; Ribnicky, D. M.; Yang, H.; Poulev, A.; Ma, J.; Kennelly, E. J.; Raskin, I. Phytochemistry 2006, 67, 1539-1546.
(13) Govorko, D.; Logendra, S.; Wang, Y.; Esposito, D.; Komarnytsky, S.; Ribnicky, D.; Poulev, A.; Wang, Z.; Cefalu, W. T.; Raskin, I. Am. J. Physiol. Endocrinol. Metab. 2007, 293, E1503-1510.
(14) Kheterpal, I.; Scherp, P.; Kelley, L.; Wang, Z.; Johnson, W.; Ribnicky, D.; Cefalu, W. T. Nutrition 2014, 30, S43-51.
(15) Obanda, D. N.; Ribnicky, D. M.; Raskin, I.; Cefalu, W. T. Nutrition 2014, 30, S59-66.
(16) Vandanmagsar, B.; Haynie, K. R.; Wicks, S. E.; Bermudez, E. M.; Mendoza, T. M.; Ribnicky, D.; Cefalu, W. T.; Mynatt, R. L. Diabetes Obes. Metab. 2014, 16, 728-738.
(17) Gertsch, J. Planta Med. 2011, 77, 1086-1098.
(18) Reddy, A. S.; Zhang, S. Expert Rev. Clin. Pharmacol. 2013, 6, 1-13.
(19) Dietz, B. M.; Chen, S.-N.; Alvarenga, R. F. R.; Dong, H.; Nikolic, D.; Biendl, M.; van Breemen, R. B.; Bolton, J. L.; Pauli, G. F. J. Nat. Prod. 2017, 80, 2284-2294.
(20) Ramos Alvarenga, R. F.; Friesen, J. B.; Nikolic, D.; Simmler, C.; Napolitano, J. G.; van Breemen, R.; Lankin, D. C.; McAlpine, J. B.; Pauli, G. F.; Chen, S. N. J. Nat. Prod. 2014, 77, 2595-2604.
(21) Ribnicky, D. M.; Roopchand, D. E.; Poulev, A.; Kuhn, P.; Oren, A.; Cefalu, W. T.; Raskin, I. Nutrition 2014, 30, S4-10.
(22) De Vincenzi, M.; De Vincenzi, A.; Silano, M. Fitoterapia 2004, 75, 615-618.
(23) van den Berg, S. J.; Punt, A.; Soffers, A. E.; Vervoort, J.; Ngeleja, S.; Spenkelink, B.; Rietjens, I. M. Chem. Res. Toxicol. 2012, 25, 2352-2367.
(24) Bojsen-Møller, K. N.; Lundsgaard, A. M.; Madsbad, S.; Kiens, B.; Ho1st, J. J. Diabetes 2018, 67, 2129-2136.
(25) Jung, S. H.; Jung, C. H.; Reaven, G. M.; Kim, S. H. Diabetologia 2018, 61, 681-687.
(26) Kryshak, E. J.; Butler, P. C.; Marsh, C.; Miller, A.; Barr, D.; Polonsky, K.; Perkins, J. D.; Rizza, R. A. Diabetes 1990, 39, 142-148.
(27) Kirk-Ballard, H.; Wang, Z. Q.; Acharya, P.; Zhang, X. H.; Yu, Y.; Kilroy, G.; Ribnicky, D.; Cefalu, W. T.; Floyd, Z. E. PLoS One 2013, 8, e57112.
(28) Eisenman, S. W.; Poulev, A.; Struwe, L.; Raskin, I.; Ribnicky, D. M. Fitoterapia 2011, 82, 1062-1074.
(29) Schmidt, B.; Ribnicky, D. M.; Poulev, A.; Logendra, S.; Cefalu, W. T.; Raskin, I. Metabolism 2008, 57, S3-9.
(30) Zuberi, A. R. Metabolism 2008, 57, S10-S15.
(31) Boudreau, A.; Cheng, D. M.; Ruiz, C.; Ribnicky, D.; Allain, L.; Brassieur, C. R.; Turnipseed, D. P.; Cefalu, W. T.; Floyd, Z. E. Nutrition 2014, 30, S11-16.
(32) Wang, Z. Q.; Floyd, Z. E.; Qin, J.; Liu, X.; Yu, Y.; Zhang, X. H.; Wagner, J. D.; Cefalu, W. T. Diabetes 2009, 58, 1488-1498.

### Example 3

Artemisia dracunculus contains, among other chalcones, two isomers of 2,4-(D)ihydroxy-4-(M)ethoxy-dihydro(C)halcone, which were thus designated as DMC-1 (compound 1) and DMC-2 (compound 2).

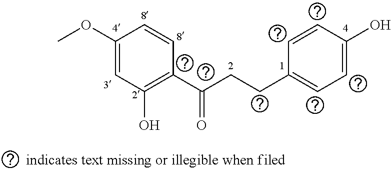 [SMILES: COc1ccc(C(=O)CCc2ccc(O)cc2)c(O)c1]

(1) DMC-1 (2′,4-dihydroxy-4′-methoxydihydrochalcone)=4′-O-Methyldavidigenin

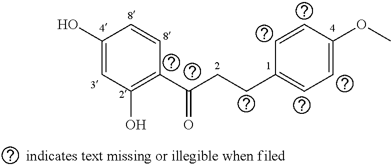 [SMILES: COc1ccc(CCC(=O)c2ccc(O)cc2O)cc1]

(2) DMC-2 (2′,4′-dihydroxy-4-methoxydihydrochalcone)=4-O-Methyldavidigenin

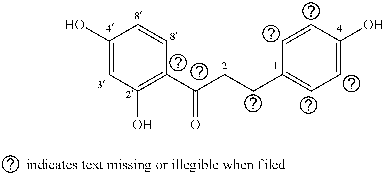 [SMILES: O=C(CCc1ccc(O)cc1)c1ccc(O)cc1O]

Davidigenin

### Example 4

An Ethanolic Extract of Artemisia dracunculus L. Activates LKB1-Mediated AMPK Signaling in Skeletal Muscle

An ethanolic extract Artemisia dracunculus L. (termed PMI-5011) improves glucose homeostasis by enhancing insulin action, and reduces ectopic lipid accumulation and metabolic inflexibility while increasing fat oxidation in skeletal muscle tissue in obese insulin resistant male mice. Bioactive DMC-2 in PMI-5011 was confirmed as a major compound that enhances insulin signaling and activation of AKT-pathway, thus playing an important role in PMI-5011 action on glucose metabolism. However, the mechanism through which PMI-5011 improves lipid metabolism is not known. AMPK is the cellular energy and metabolic sensor and a key regulator of lipid metabolism in muscle. We examined whether PMI-5011 enhances activity of AMPK signaling pathway using murine C2C12 muscle cell culture and skeletal muscle tissue from C57BL6 mice. We found that PMI-5011 increases Thr172-phosphorylation of AMPK in a LKB1-dependent manner in muscle cells and skeletal muscle tissue, however hepatic AMPK activation by PMI-5011 was not observed. Increased AMPK activity by PMI-5011 affects downstream signaling of AMPK, resulting in inhibition of ACC and increased SIRT1 protein levels. Using DESIGNER technology, we removed DMC-2 from the PMI-5011 extract and found that compounds other than DMC-2 are responsible for AMPK activation and its downstream effects. Compared to AICAR and metformin, PMI-5011 appears to be a more efficient activator for AMPK in muscle cells. In conclusions, PMI-5011 through AMPK activation and downstream inhibition of ACC, regulates lipid metabolism by increasing fat oxidation and decreasing lipid synthesis. Thus, the AMPK-activating potential of PMI-5011 adds therapeutic value to PMI-5011 or its constituent compounds in treating insulin resistance or type 2 diabetes.

Introduction

The 5-AMP-activated protein kinase (AMPK) is a master regulator of metabolism that is activated in low cellular energy conditions (increased AMP/ATP ratio) to switch on ATP-generating, catabolic pathways and switch off energy-demanding, ATP-consuming processes. AMPK functions as a serine/threonine kinase composed of a catalytic α, and regulatory β and γ subunits. AMP binding to the γ subunit allows AMPK to sense shifts in AMP/ATP ratio and causes a conformational change that exposes the activating phosphorylation site at Thr172 of the catalytic a subunit (1,2). AMPK activation through phosphorylation at Thr172 on the α-subunit is regulated independently by two upstream kinases, liver kinase B1 (LKB1) and Ca⁽²⁺⁾/calmodulin-dependent protein kinase kinase-β (CaMKKβ). Increased AMP levels stimulate LKB1-mediated phosphorylation, whereas increased intracellular calcium activates CaMKKβ-mediated phosphorylation (1,3). Once activated by Thr172 phosphorylation, AMPK coordinates the activities of key metabolic pathways that control lipid and glucose metabolism, protein synthesis, cell growth and proliferation, autophagy, mitochondrial biogenesis and function (4). Given the central role of AMPK in monitoring cellular energy availability, its impact on metabolism is important in tissues that undergo highly dynamic changes in energy demand, such as skeletal muscle.

Skeletal muscle is unique in its ability to vary its metabolic rate in response to dynamic changes in physical activity or metabolic states. It is responsible for 70-90% insulin-stimulated glucose uptake during feeding and is a predominant site for fatty acid oxidation during fasting or exercise because of its high-energy demands and large mass (5-7). AMPK-dependent regulation of ATP availability is critical to maintaining the metabolic flexibility necessary to switch between glucose and fatty acid oxidation in response to different physiological states such as fed and fasted or activity states ranging from resting to vigorous exercise.(8). The inability to adapt to these changes in skeletal muscle is associated with insulin resistance and metabolic disorders (9,10). Impaired signaling in response to insulin in skeletal muscle, coupled with accumulation of ectopic lipid metabolites, reduced fatty acid oxidation and impaired mitochondrial function plays a pivotal role in developing obesity-associated metabolic disorders such as insulin resistance and type 2 diabetes (11-13). Thus, a strategy focused on activating AMPK signaling in skeletal muscle can have a therapeutic use as a monotherapy or in combination with other approaches to treat obesity-associated metabolic dysfunction.

There is a long history of pharmacological use of natural products for their anti-diabetic effects. This includes Metformin, a widely used synthetic biguanide based on compounds found in Galega officinalis L., commonly known as French lilac (14). Metformin activates AMPK in an LKB1 dependent manner that is associated with reduced hepatic glucose output and improved glucose uptake in skeletal muscle (15,16) although neither protein is a direct target of Metformin (17). Artemisia dracunculus L. or Russian tarragon is an herb that historically has been used in traditional medicine as an anti-inflammatory and antioxidant agent (18), but also for its anti-diabetic properties (18). An ethanolic extract of A. dracunculus, termed PMI-5011 has been investigated in rodent and cell culture models because of its antidiabetic effect (19-21). Using bioactivity-guided fractionation method, five compounds were identified in PMI-5011 as having anti-diabetic effects (22,23). In a series of experiments, we showed that PMI-5011 improves glucose metabolism by reducing blood glucose and insulin levels and enhancing insulin signaling in skeletal muscle in obese, insulin resistant mice (24-26). The hypoglycemic activity of PMI-5011 is primarily attributed to 2′,4′-dihydroxy-4-methoxydihydrochalcone (DMC-2, 4-O-methyldavidigenin) and to a lesser extent, its regioisomer, 2′,4-dihydroxy-4′-methoxydihydrochalcone (DMC-1, 4′-O-methyldavidigenin) (27). In experiments using a recently developed DESIGNER (Deplete and Enrich Select Ingredients to Generate Normalized Extract Resources) approach, DMC-2 and DMC-1 were selectively removed from the extract to definitively determine if DMC-2 and DMC-1 account for enhanced insulin signaling in skeletal muscle (28). Using this approach in an animal model of obesity-induced insulin resistance, we confirmed that DMC-2 (along with DMC-1) is the major phytochemical regulating insulin signaling in skeletal muscle and lowering blood glucose levels. The impact of DMC-2 on blood glucose levels was comparable to Metformin (28). Interestingly, phosphorylation of AS160 occurred in the complete extract and the “knock-out” extract (KOE) containing all components of PMI-5011 other than DMC-2 and DMC-1, although AKT phosphorylation did not occur in the KOE (28). Since AS160 is also activated by AMPK, PMI-5011 and the KOE activate the AMPK-signaling pathway in skeletal muscle. Recent studies demonstrate that PMI-5011 supplementation results in favorable changes in lipid metabolism by increasing mitochondrial fatty acid oxidation, improving metabolic flexibility in skeletal muscle, and decreasing triglyceride (TG) content in the skeletal muscle and liver of obese, insulin resistant mice without changes in body weight or adiposity (26,29,30). In the current study, we used a high fat diet-fed mouse model of obesity-related insulin resistance and murine C2C12 skeletal muscle cells to test PMI-5011's effect on AMPK activation. Herein we demonstrate that PMI-5011 increases phosphorylation of AMPK at Thr172 in C2C12 myotubes and skeletal muscle, but not the liver, of obese mice. The KOE fraction of PMI-5011 contains the major bioactive that enhances AMPK-signaling in muscle, independent of DMC-2 and DMC-1. We also determined that phosphorylation of LKB1, the upstream activator of AMPK, is increased with PMI-5011 treatment. However, blocking CaMMKβ-kinase, the other upstream activator of AMPK, did not affect PMI-5011 action on AMPK-signaling pathway, indicating PMI-5011 promotes AMPK activation in an AMP-dependent manner. In addition, we report changes in activities of pathways that are downstream targets of AMPK-signaling such as ACC and SIRT1, indicating that PMI-5011 regulates lipid metabolism in skeletal muscle by activating the AMPK pathway.

Methods

Sourcing and Characterization of PMI-5011 Extract

Artemisia dracunculus was grown in a Rutgers University (New Brunswick, N.J.) greenhouse under uniform and strictly controlled conditions using commercially available seeds (Sheffield's Seed Company, Locke, N.Y.). Preparation of the ethanolic extract from A. dracunculus (PMI-5011), detailed information about quality control, and biochemical characterization were previously reported (22,23,28,31-34). Bioactivity guided fractionation using in vitro bioassays followed by confirmation in vivo, identified five bioactive compounds (22). The Knock-out extract (KOE) lacking DMC-1 and DMC-2 and the enriched DMC-2 fraction were generated by the Center of Natural Products Technologies (CENAPT) (University of Illinois at Chicago) using DESIGNER technology (28). DMC-2 was synthesized as described (32).

Experimental Animals

Male C57BL/6J mice were obtained from Jackson Laboratories (Bar Harbor, Me., USA). All animal experiments were approved by the Rutgers University Animal Care and Use Committee (Protocol #04-023) and were in compliance with the NIH Guide for the Care and Use of Laboratory. The animals were housed singly with a 12 hour light-dark cycle at 24° C. At 4 weeks of age, the mice were placed on a very high fat diet (60 kcal % fat, D12492, Research Diets) for 16 weeks. Body weight was measured weekly. The mice were acclimated to gavage over a 2-week period prior to administering the vehicle control (Labrasol), PMI-5011 extracts, synthetic DMC-2, or metformin via gavage. Blood glucose levels were assayed at baseline and each hour thereafter up to 6 hours post gavage. The mice were euthanized and tissues were collected for analysis at 6 hours after gavage.

Skeletal Muscle Cell Culture

Murine C2C12 myoblasts cells were obtained from the American Type Culture Collection (#CRL-1458) and cultured in Dulbecco's modified Eagle's medium (DMEM), high glucose (25 mM) with 10% fetal bovine serum, 2 mM glutamine, and antibiotics (100 units/mL penicillin G and 100 μg/mL streptomycin), in a humidified chamber at 37° C. and 5% CO₂. To obtain fully differentiated myotubes, the medium was exchanged for DMEM, high glucose with 2% horse serum, glutamine, and antibiotics, when the myoblasts reached confluence. The medium was replaced every 48 hours, and the cells were maintained in this medium until fully differentiated, when the medium was exchanged for DMEM, low glucose (5 mM) with 2% horse serum. The myotubes were fully formed by the fourth day post-induction. The experiments analyzing the impact of each experimental material on insulin signaling in the presence of palmitate-induced insulin resistance were performed as described previously (26). After starvation, cells were treated overnight with PMI-5011, KOE, enriched DMC-2/DMC-1 fraction, or synthetic DMC-2, all at 10 μg/ml, solved in DMSO as indicated in the text or figures. The myotubes were also treated FK-506 (10 μM, Cayman), STO-609 (10 μg/ml, Fisher Scientific), 5-Aminoimidazole-4-carboxamide ribonucleotide (AICAR, 0.5 mM, Sigma), metformin (2 mM, Sigma), or MG132 (10 μM, Boston Biochem) as indicated.

For siRNA transfection, C2C12 cell culture was carried out in a 24-well plate format. At 100% confluence, the growth medium was replaced by differentiation medium with 2% horse serum. After 3 days, fully differentiated myotubes were transfected with AMPKα1 siRNA (sc-29674, Santa Cruz) or AMPKα2 siRNA (sc-38924, Santa Cruz) using Dharmafect Duo Transfection Reagent (Thermo Scientific, Cat #T-2010-02) according to the manufacturer's instruction. Myotubes were harvested at 24 or 48 hours after the transfection. Sixteen hours prior to harvesting the cells, the vehicle (DMSO) or PMI-5011 (10 μg/ml) were added to the differentiation medium.

Protein Expression Analysis

Skeletal muscle and liver tissue lysates were prepared from powdered tissue by homogenization in 25 mM HEPES, pH 7.4, 1% Igepal CA630, 137 mM NaCl, with 10 mM Na4P2O7, 100 mM NaF, 2 mM Na3VO41 mM PMSF, 10 μg/mL aprotinin, 1 μg/mL pepstatin, and 5 μg/mL leupeptin. C2C12 myotube lysates were prepared by sonication in 20 mM Tris pH 7.4, 1% Igepal CA630, 5 mM EDTA with 10 mM Na4P2O7, 100 mM NaF, 2 mM Na3VO4, 1 mM PMSF, 10 μg/ml aprotinin, 1 μg/ml pepstatin, and 5 μg/ml leupeptin. The tissue and cell lysates were centrifuged at 14,000×g for 10 minutes at 4° C. Protein concentrations were determined using BCA assays (Thermo Fisher Scientific, Rockford, Ill., USA) and the resulting supernatants (50 μg) were resolved by 10% SDS-PAGE and subjected to immunoblotting. Nitrocellulose membranes were incubated with antibodies for 1-2 hours at RT or overnight at 4° C. Image J software was used for quantification of the western blot band intensities.

Statistical Analysis

Data are presented as the mean±standard deviation of results from three independent experiments. The Student's t-test was used to determine the significance of differences between two groups. Differences with a p value <0.05 were regarded as being statistically significant and denoted using an asterisk. All graphs were generated using GraphPad Prism 8.4.

Results

PMI-5011 is a complex mixture of bioactive phytochemicals. DMC-2 (and DMC-1) is the principle phytochemical responsible for enhanced insulin signaling in skeletal muscle and the glucose lowering effect of the PMI-5011 extract. Despite this, DMC-2 does not account for all of the detectable biological activities present in the extract. For example, botanical extracts containing multiple phytochemicals can act on multiple pharmacological targets, where each compound could affect different cellular pathways independently or in combination to impact cellular function (35). Moreover, several of the identified bioactive compounds in PMI-5011 are present in skeletal muscle cell lysates along with DMC-2 (28).

31.7% of the PMI-5011 by weight is the enriched DMC-2 fraction containing DMC-2 and DMC-1 (Table 1). The knock-out extract (KOE) fraction generated by removing DMC-2 and DMC-1 contains davidigenin, 6-demethoxycapillarisin and sakuranetin that altogether constitutes about 68.3% of the PMI-5011 (Table 1). The volatile elemicin and iso-elemicin are plentiful methoxyallylbenzenes in Russian tarragon and, in the original PMI-5011 as well. However, due to the inevitable solvent evaporation during the preparation of dried extract and KOE, elemicin and isoelemicin are significantly decreased in the KOE fraction (Table 1) (28).

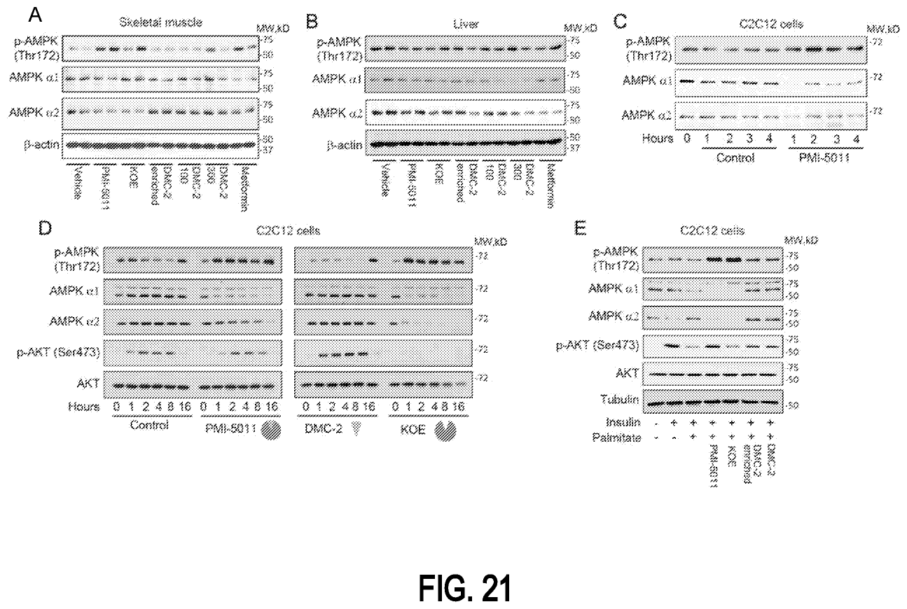

PMI-5011 upregulates AMPK-signaling in skeletal muscle, but not in the liver of obese mice. Previous studies revealed that PMI-5011 supplementation increased mitochondrial fatty acid oxidation and reduced intracellular triglyceride levels in skeletal muscle of diet-induced obese male and female mice (26,30). Moreover, experiments in vitro using skeletal muscle homogenates from high fat fed male and female mice indicated PMI-5011 supplementation enhanced the ability of skeletal muscle to switch between fat and carbohydrate oxidation, an indicator of metabolic flexibility (26,30). Based on these results, and given that AMPK is a key metabolic sensor, we asked if PMI-5011 activates AMPK-signaling in skeletal muscle. Activation of AMPK was evaluated by phosphorylation at Thr172 on the α-catalytic subunit of AMPK, and PMI-5011 supplementation enhanced AMPK phosphorylation in skeletal muscle in high fat-fed obese mice compared to vehicle-supplemented control mice (FIG. 21, panel A. Notably, the increase in AMPK phosphorylation in skeletal muscle tissue from mice supplemented with KOE only, a fraction of PMI-5011 lacking DMC-1 and DMC-2, was comparable to the level in mice with PMI-5011 supplementation. However, AMPK phosphorylation in skeletal muscle was not changed from vehicle (Labrasol) gavage when the mice were supplemented with enriched DMC-2, a fraction of PMI-5011 containing only DMC-1 and DMC-2, or synthetic DMC-2 (at 100 μg/kg or 300 μg/kg body weight). Interestingly, the increase in AMPK phosphorylation induced by PMI-5011 and the KOE fraction was similar to the phosphorylation level of AMPK by metformin, a well-known and clinically important AMPK-activator.

We previously reported that PMI-5011 supplementation significantly reduced liver triglyceride content in HFD-fed male mice, but not in HFD-fed female mice (26,30). For that reason, we also examined the effect of PMI-5011 supplementation on hepatic AMPK activation. Notably, phosphorylation of AMPK at Thr172 in liver was not changed by treatment with PMI-5011, the KOE fraction, enriched DMC-2 fraction or the synthetic DMC-2 (FIG. 21, panel B).

Taken together, our data demonstrate that PMI-5011 stimulates phosphorylation of AMPK at Thr172 in skeletal muscle, but not in the liver of high fat-fed obese mice. Also, the KOE fraction mediates PMI-5011 action on activating AMPK in skeletal muscle, while enriched DMC-2 fraction has no effect on phosphorylation of AMPK, at least under basal condition.

PMI-5011 increases AMPK-signaling in muscle cells. To understand the mechanism of PMI-5011 action on AMPK signaling pathway in skeletal muscle, we used the murine C2C12 skeletal muscle cell culture model. First, we determined whether PMI-5011 treatment enhances activity of AMPK pathway in C2C12 myotubes. Notably, phosphorylation of AMPK at Thr172 was increased in myotubes treated with PMI-5011 for up to 4 hours (FIG. 21, panel C). Importantly, the AMPK activating effect of PMI-5011 was present in myotubes when cells were exposed to PMI-5011 for up to 16 hours (FIG. 21, panel D). Increased phosphorylation of AMPK was observed in KOE-treated myotubes, but not in myotubes exposed to enriched DMC-2, which was consistent with skeletal muscle tissue from the obese mice (FIG. 21, panel A and D). In agreement with the previous data (28), phosphorylation of AKT was preserved in myotubes exposed to enriched DMC-2, however, it was lost in KOE-treated myotubes (FIG. 21, panel D). Most important, the enhanced phosphorylation level of AMPK was noticeable at 1 hour of the PMI-5011 treatment compared to the level in control myotubes.

To mimic lipid-induced insulin resistant condition in vitro, C2C12 myotubes were treated with palmitate for 16 hours, followed by insulin for 10 minutes prior to cell collection. Consistent with the in vivo data, phosphorylation of AMPK was increased in myotubes treated with PMI-5011 and KOE in the presence of insulin and palmitate (FIG. 21, panel E). Interestingly, a slight increase in phosphorylation of AMPK was observed in myotubes treated with enriched DMC-2 and synthetic DMC-2. In agreement with our earlier findings (28), PMI-5011 increased insulin stimulated AKT phosphorylation and this effect was mediated by enriched DMC-2 and synthetic DMC-2, whereas, KOE treatment alone did not change insulin stimulated AKT phosphorylation in myotubes (FIG. 21, panel E).

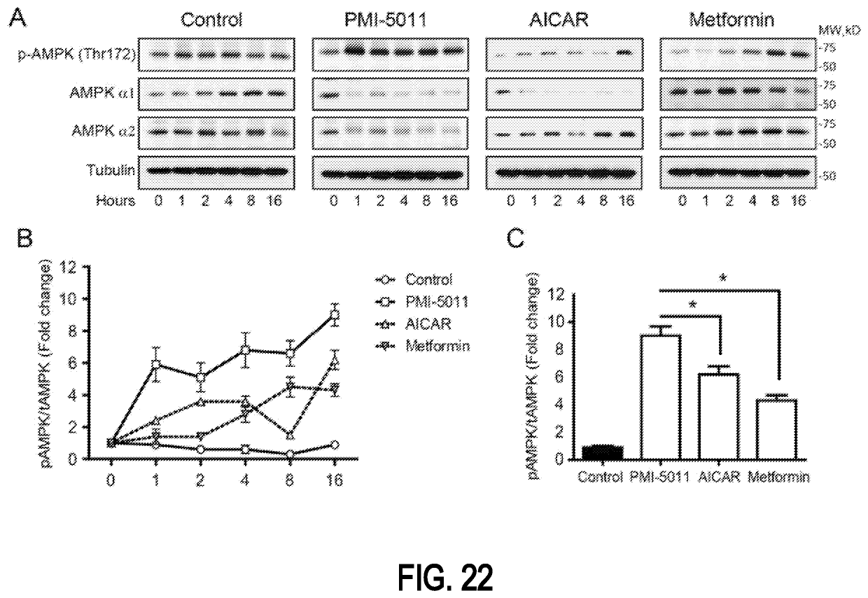

Next, the effect of PMI-5011 on AMPK activation in myotubes was compared to the other well-known AMPK activators such as AICAR and metformin (FIG. 22, panel A). Activation of AMPK was evaluated as a ratio of phosphorylated portion of AMPK at Thr172 on a subunits to total AMPK a subunits, where protein expression of α1 and α2 subunits were combined and presented as total AMPK (FIG. 22, panel B) The effect of PMI-5011 on AMPK activation appeared very early and the 6-fold increase in phosphorylation of AMPK was observed in myotubes as early as 1 hour of exposure to PMI-5011, and continued to increase (FIG. 22, panels A and B). However, increased phosphorylation of AMPK by AICAR and metformin was delayed compared to PMI-5011, and the increase was only 2.4- and 1.4-fold at 1 hour of the treatment, respectively. In myotubes exposed to AMPK-activators for 16 hours, the level of AMPK-phosphorylation by PMI-5011 was significantly higher compared to the levels achieved by AICAR and metformin (FIG. 22, panel C).

Thus, we demonstrate here that the positive effects of PMI-5011 on activation of AMPK and AKT pathways observed in skeletal muscle tissue, are reproducible in the muscle cell model. Specifically, KOE is the fraction that mediates PMI-5011's effect on activating AMPK signaling pathway, whereas, DMC-2 mediates PMI-5011's effect on insulin stimulated activation of AKT in skeletal muscle cells. Moreover, in skeletal muscle cells, PMI-5011 appears to be a more efficient AMPK-activator than AICAR or metformin.

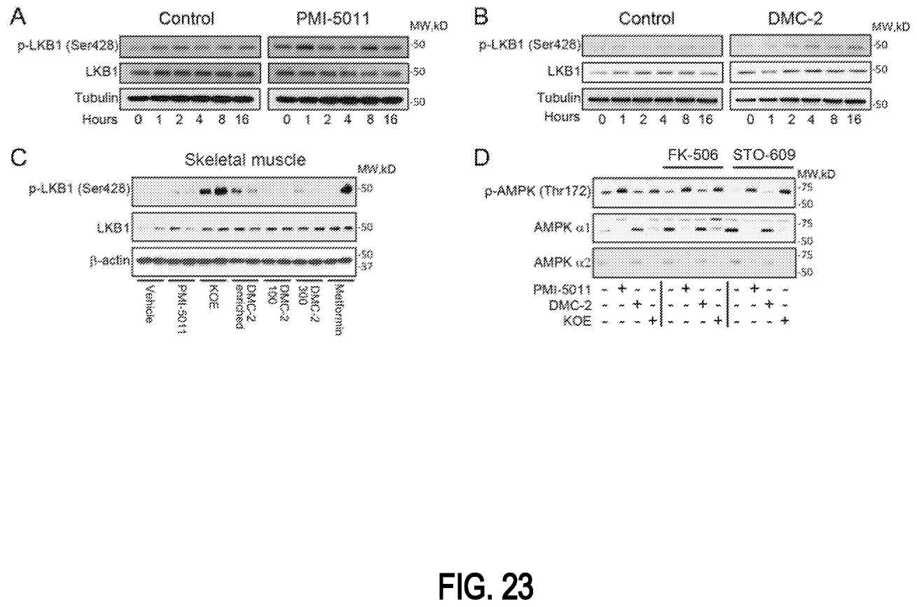

LKB1 kinase mediates PMI-5011 effect on activation of AMPK-pathway. Two upstream kinases, liver kinase B1 (LKB1) and Calcium/calmodulin-dependent kinase kinase beta (CaMKKβ) phosphorylate Thr172 of the a subunits of AMPK (1). To determine whether these upstream kinases are involved in action of PMI-5011 on enhancing the phosphorylation of AMPK, we used C2C12 myotubes. LKB1 activity was evaluated by its phosphorylation at Ser428, which is required for nuclear export of LKB to the cytoplasm (36,37) where it interacts with AMPK. PMI-5011 treatment increased the phosphorylation of LKB1 in myotubes and the increase was apparent after 1-hour exposure to PMI-5011 (FIG. 23, panel A), which was consistent with the phosphorylation profile of AMPK in myotubes treated with PMI-5011 (FIG. 22, panel B). However, phosphorylation of LKB1 was only slightly increased at the end of 16-h exposure when myotubes were treated with enriched DMC-2 (FIG. 23, panel B). This data indicates that KOE might be the fraction of PMI-5011 that is responsible for enhanced phosphorylation of LKB1 in myotubes starting as early as 1-hr exposure to PMI-5011.

To determine if KOE activates LKB1, we examined LKB1 phosphorylation at Ser428 in skeletal muscle tissue. In agreement with the data in myotubes, LKB1-phosphorylation was increased in skeletal muscle when mice were administered PMI-5011 total extract compared to vehicle gavaged mice (FIG. 23, panel C). Phosphorylation of LKB1 Ser428 was markedly increased in skeletal muscle of KOE-treated mice and this level was comparable to the level achieved by administering metformin Although a modest increase in phosphorylation of LKB1 was detected in skeletal muscle tissue when mice were treated with the enriched DMC-2 fraction, the levels of LKB1-phosphorylation observed with synthetic DMC-2 treatment were not different from the level detected in the skeletal muscle tissue of vehicle-treated mice. As a separate pathway to activate AMPK, CaMKKβ binds to AMPK through a direct interaction of their kinase domains (38). FK-506 and STO-609 are small molecules that selectively inhibit CaMKKβ by blocking the binding (39,40). Notably, the presence or absence of both inhibitors of CaMKKβ did not change the phosphorylation profiles of AMPK in myotubes treated with PMI-5011, and its fractions enriched DMC-2 and KOE (FIG. 3D). This result indicates CaMKKβ kinase is not involved in AMPK activation by PMI-5011, specifically by KOE.

Taken together, our data indicates LKB1, but not CaMKKβ is the upstream kinase that phosphorylates AMPK in myotubes treated with PMI-5011 and skeletal muscle in mice supplemented with PMI-5011. Consistent with the AMPK phosphorylation data, KOE is the fraction of the PMI-5011 extract responsible for PMI-5011-mediated LKB1 activation.

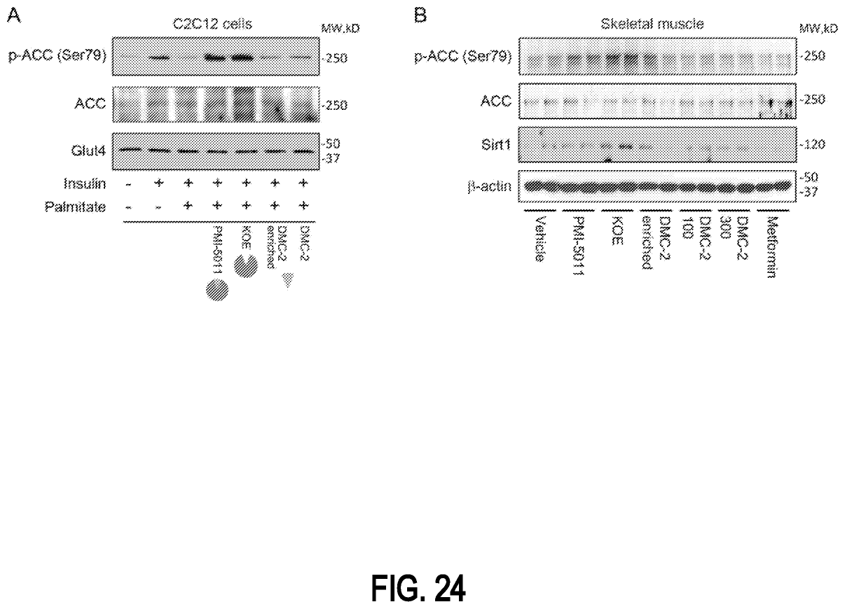

PMI-5011 and KOE modulate downstream events of AMPK activation to link PMI-5011 and KOE to cellular metabolism in skeletal muscle. AMPK activation increases catabolic activities to generate energy and decreases anabolic activities that consume energy, thus AMPK integrates energy demands with cellular metabolic pathways. AMPK regulates fatty acid metabolism through acetyl-CoA carboxylase (ACC) that has two isoforms ACC1 and ACC2 (37). ACC catalyzes the carboxylation of acetyl-CoA to produce malonyl-CoA, leading to increased fatty acid synthesis and inhibition of fatty acid oxidation (41). AMPK inhibits ACC by directly phosphorylating both isoforms of ACC, thus activation of AMPK reduces lipid synthesis and enhances fatty acid oxidation. To determine whether PMI-5011 modulates activity of ACC through AMPK-activation, we examined phosphorylation of ACC at Ser79, the AMPK target site. Notably, phosphorylation of ACC was enhanced in myotubes treated with PMI-5011 and as well as KOE in the presence of insulin and palmitate compared to the level of the phosphorylation in the control myotubes in the presence of insulin and palmitate (FIG. 24, panel A). However, ACC phosphorylation was unaffected in myotubes treated with enriched DMC-2 and synthetic DMC-2. These data indicate that activation of AMPK by PMI-5011 and specifically by KOE inhibits ACC activity, leading to reduced fatty acid synthesis and increased mitochondrial fatty acid oxidation in skeletal muscle.

AMPK also enhances the activity of the NAD-dependent deacetylase Sirtuin 1 (SIRT1) by increasing cellular NAD+ levels or the NAD+/NADH ratio (42). We previously demonstrated that PMI-5011 increases protein expression of SIRT1 in skeletal muscle but not in the liver of obese mice (26). In this study, we found that protein expression of SIRT1 was increased noticeably in skeletal muscle of mice administered KOE, whereas SIRT1 expression in skeletal muscle of mice treated with enriched DMC-2 or synthetic DMC-2, was comparable to the level of SIRT1 in muscle tissue of vehicle-treated mice (FIG. 24, panel B). Although, recent reports state that metformin as a direct SIRT1-activating compound using computational modeling and cell-free assays (43), we did not observe an increase in SIRT1 protein expression in skeletal muscle of the metformin treated mice (FIG. 24, panel B).

Altogether, our data indicate that the KOE fraction of PMI-5011 activates AMPK to downregulate ACC, leading to inhibition of ACC-mediated anabolic pathways in skeletal muscle and stimulation of ACC2-mediated catabolic pathways such as fat breakdown by oxidation in the mitochondria. In line with cellular energy savings, our data also show the KOE fraction alters SIRT1 protein levels.

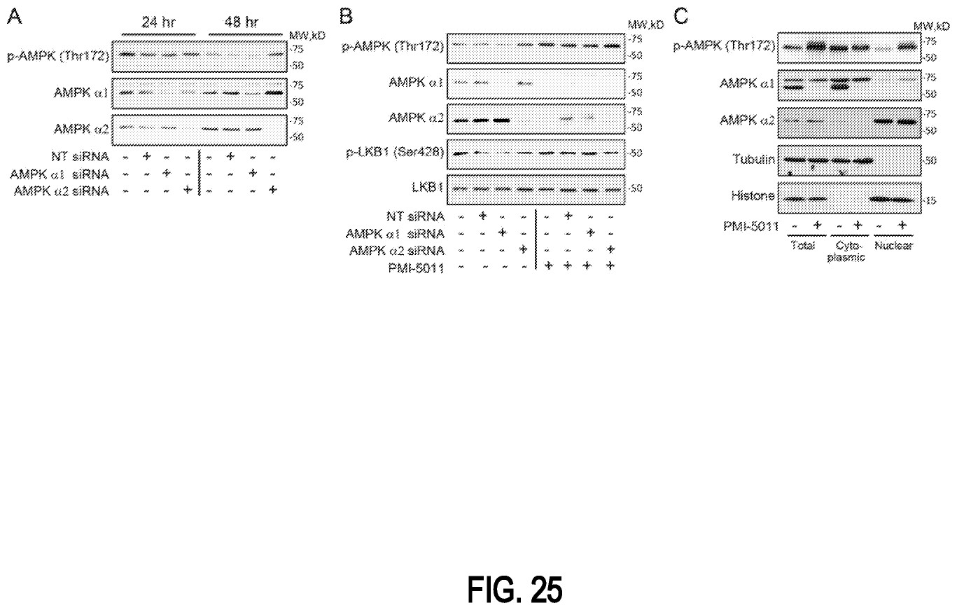

Both isoforms of alpha-subunit of AMPK are involved in PMI-5011 action in myotubes. The AMPK α catalytic subunit is encoded by two genes (α1 and α2) that have tissue-specific expression patterns. The α1 isoform is ubiquitously expressed while the α2 subunit is highly expressed in skeletal and cardiac muscle, and at low levels in other tissues (44). Previously we reported that PMI-5011 supplementation did not affect gene expression of AMPKα1 and AMPKα2 in skeletal muscle of mice (26). However, functional differences between two isoforms of catalytic subunits of AMPK have been reported. Particularly in skeletal muscle, AMPKα1 has been shown to control mTORC1 signaling, and thus controls muscle cell size, while AMPKα2 mediates muscle metabolic adaptation (45). To determine whether PMI-5011 acts on AMPK-activation in an isoform-specific manner, we used siRNA to specifically deplete AMPKα1 or α2 in C2C12 myotubes. Protein expression of AMPKα1 and AMPKα2 in siRNA-treated myotubes was depleted after 24 hours and remained markedly reduced after 48 hours (FIG. 25, panel A). Basal phosphorylation of AMPK with the nonspecific siRNA was observed at 24 hours, however, this level was lower at 48 hours. On the other hand, the PMI-5011 induced phosphorylation of AMPK was enhanced compared to the control even after 48 hours (FIG. 25, panel B). In addition, PMI-5011 induced phosphorylation of LKB1 Ser428 was still observed at 48 hours. Interestingly, individually depleting α1 or α2 did not affect AMPK phosphorylation, indicating that both isoforms contribute to PMI-5011-mediated AMPK activation.

In mammalian cells, AMPK signaling is compartmentalized with activation of different pools of AMPK depending on various metabolic and stress conditions (46,47). The two a subunits of AMPK have a differential subcellular localization pattern. AMPKα1 is detected in the cytoplasm and nucleus, but AMPKα2 is localized in the nucleus (8,48). In the PMI-5011 treated C2C12 myotubes, phosphorylation of AMPK was observed in both cytoplasmic and nuclear fractions as well as total cell lysates (FIG. 25, panel C). The PMI-5011-induced increase in nuclear AMPK phosphorylation coincides with increased nuclear AMPK al, indicating PMI-5011 acts through both isoforms to affect cytoplasmic and nuclear functions of AMPK in skeletal muscle.

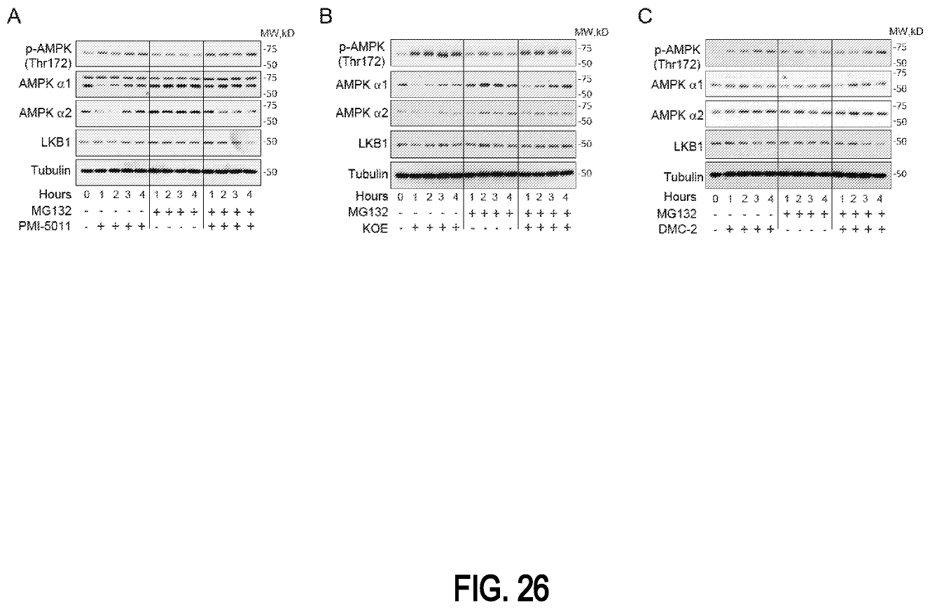

Reduced levels of AMPKα1 and α2 proteins, but not LKB1 in the myotubes treated with PMI-5011 (FIG. 25, panel B) and evidence that AMPK protein stability is regulated by the ubiquitin-proteasome system (49,50) prompted us to examine whether degradation of the AMPKα subunits is proteasome dependent. The presence of the proteasome inhibitor MG132 partially preserved α1 and α2 proteins in myotubes treated with PMI-5011 or KOE (FIG. 26, panel A and B). Interestingly, proteasome inhibition reduced AMPK phosphorylation at Thr172, indicating degradation of an unidentified inhibitor of AMPK activation is essential for AMPK activity. The impact of the inhibitor is overridden by PMI-5011 extract or the KOE fraction. Contrary to the KOE-treated myotubes, neither AMPK-phosphorylation nor AMPKα1 and AMPKα2 protein expression were changed in myotubes treated with enriched DMC-2 in the presence or absence of MG132 (FIG. 26, panel C). In addition, changes in protein expression with PMI-5011 or KOE treatments were specific to α-subunits of AMPK and did not affect protein expression of LKB1 (FIG. 26, panels A-C). Nevertheless, protein abundance of AMPKα1 and AMPKα2 in skeletal muscle of mice appears lower with KOE-supplementation, and unchanged with enriched DMC-2 supplementation, consistent with PMI-5011-mediated regulation of AMPK activity independent of DMC-2.

Taken together, these data demonstrate that compounds in the KOE from PMI-5011 activates AMPK through phosphorylation of both a subunits in skeletal muscle. PMI-5011 and the KOE fraction reduced protein abundance of AMPKα1 and AMPKα2 subunits while preserving AMPK phosphorylation. While protein levels of both isoforms of AMPKα subunit are reduced by PMI-5011 and KOE in the C2C12 myotubes, AMPKα2 subunit protein levels are predominantly affected in the skeletal muscle tissue (FIG. 21, panel A).

Discussion

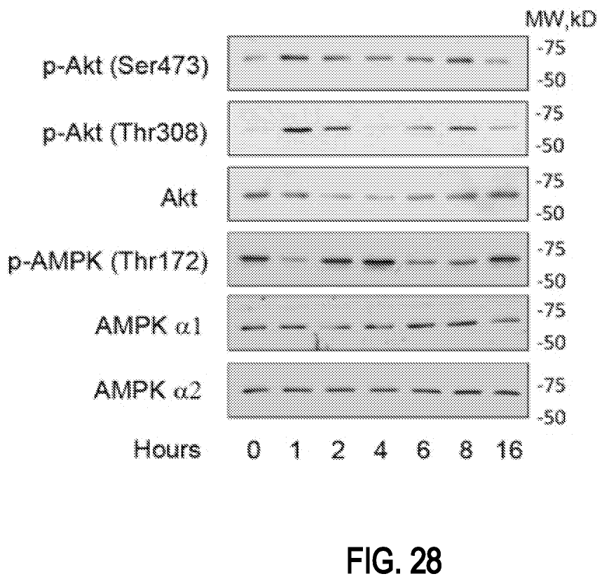

Our previous studies demonstrated the anti-diabetic effect of the ethanol-based extract from Artemisia dracunculus L., termed PMI-5011 corresponded to enhanced insulin-AKT signaling in the obese insulin resistant state, resulting in improved glucose homeostasis (19,21,25,29). When 2′,4′-dihydroxy-4-methoxydihydrochalcone (DMC-2) is selectively depleted from the PMI-5011 extract, the resulting “knock-out” extract (KOE) no longer promotes AKT activation in skeletal muscle, identifying DMC-2 as the compound responsible for AKT activation in skeletal muscle (28). However, AS160 phosphorylation is enhanced in the KOE even though AKT phosphorylation is reduced, pointing to regulation of AMPK signaling by the KOE, given that AS160 is also a downstream target of AMPK (51). This is consistent with our earlier findings that PMI-5011 promotes fatty acid oxidation and improves metabolic flexibility in skeletal muscle of high fat-fed male and female mice (26,30). In the current study, we show that the PMI-5011 KOE compounds robustly stimulate AMPK phosphorylation in skeletal muscle independent of AKT activation, leading to inactivation of ACC, a critical regulator of fatty acid oxidation. Thus, the complex mixture of phytochemicals in PMI-5011 affects both insulin signaling and energy sensing in skeletal muscle, integrating the reciprocal relationship between AKT and AMPK signaling in skeletal muscle (FIG. 28) (52). Moreover, KOE administration had no effect on hepatic AMPK activity, indicating the effect is specific to skeletal muscle.

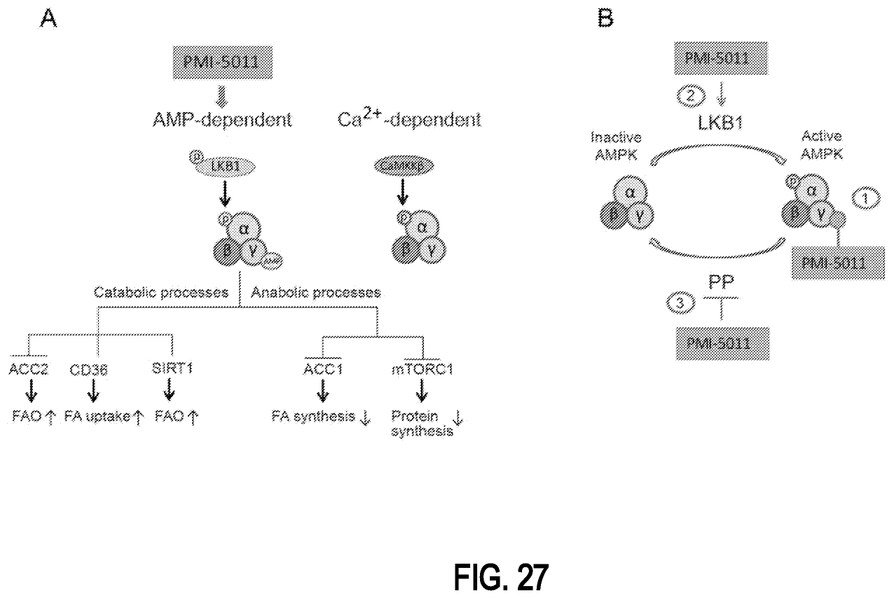

When we examined two upstream kinases that phosphorylate the AMPK alpha subunit at Thr172, we found that the energy sensing AMP-dependent liver kinase B1 (LKB1) kinase mediates the effect of PMI-5011 and KOE on AMPK activation. LKB1 links fatty acid uptake by CD36 to AMPK activation and fatty acid oxidation (53). We found that expression of CD36 was increased in skeletal muscle tissue of female mice fed a diet with PMI-5011 (30). Coupled with KOE-stimulated ACC phosphorylation and increased protein levels of the energy-sensing NAD+-dependent SIRT1 deacetylase, both substrates of AMPK that promote fatty acid oxidation when modified by AMPK (1,42,54), our results place KOE as regulating early signaling events that coordinate fatty acid uptake and oxidation (FIG. 27, panel A).

Without wishing to be bound by theory, the rapid and sustained activation of AMPK by PMI-5011 can be explained by several 1 mechanisms underlying PMI-5011 KOE-mediated AMPK activation. LKB1 dependent phosphorylation of AMPK at Thr172 is stimulated by AMP binding to the AMPK α-subunit (1), an event that is reproduced by AICAR as an AMP analog. The PMI-5011 KOE modulates AMPK Thr172 phosphorylation and AMPKα1 protein levels in a pattern similar to AICAR, indicating that one or more compounds in KOE directly bind the AMPKα subunit (FIG. 27, panel B).

Alternatively, the strong stimulation of LKB1 phosphorylation at Ser428 by KOE indicates a mechanisms by which KOE mediates LKB phosphorylation and nuclear export either directly or indirectly to affect AMPK Thr172 phosphorylation. Although PMI-5011 regulates AMPK phosphorylation of the cytoplasmic and nuclear AMPK alpha subunits, our studies do not address whether PMI-5011 or KOE affect LKB1/AMPK interaction in either cellular compartment. However, a mechanism involving metformin-mediated LKB1 phosphorylation at Ser428 via an atypical protein kinase C zeta (αPKCζ) (55) that is regulated by metformin in the skeletal muscle of diabetic subjects is indicated (56). The αPKCζ-dependent LKB1 Ser428 (399 short form) phosphorylation promotes LKB nuclear export (36) and both KOE and metformin stimulate LKB1 Ser428 phosphorylation in skeletal muscle, indicating that KOE acts via αPKCζ to indirectly affect AMPK activity. However, while KOE mirrors the effect of metformin on AMPK Thr172 phosphorylation, metformin does not alter AMPKζ1 protein levels, indicating KOE has properties related to AMPK activation that are distinct from metformin (FIG. 27, panel B).

Without wishing to be bound by theory, a third mechanism is related to the sustained effect of PMI-5011 and KOE on AMPK Thr172 phosphorylation even though both PMI-5011 and KOE stimulate proteasome-dependent degradation of the AMPK alpha subunits. We found that AMPK Thr172 phosphorylation is inhibited when proteasome activity is blocked, indicating a factor responsible for AMPK Thr172 dephosphorylation is degraded by the proteasome. This effect is overridden by either PMI-5011 or KOE, consistent with our previous report showing a significant decrease in phosphatase activity in skeletal muscle of genetically obese mouse model supplemented with PMI-5011 (25) (FIG. 27, panel B).

Without wishing to be bound by theory, the multiple mechanisms of action of PMI-5011 and KOE effect on AMPK activation reflect the mixture of compounds present in the KOE. Of the four phytochemicals present in the KOE that have anti-diabetic activity (22), sakuranetin and 6-demethoxycapillarisin are interesting as a regulator of AMPK activity. Sakuranetic is a flavonoid derived from naringenin, a compound found to improve insulin sensitivity, skeletal muscle glucose uptake and mitochondrial function through AMPK phosphorylation (57-59) and can directly bind AMPK (60). 6-demethoxycapillarisin is related to coumarins, a class of phytochemicals that activate AMPK signaling via increased phosphorylation of Thr172 in skeletal muscle and adipocytes (61,62). Thus, both sakuranetin and 6-demethoxycapillarisin are candidate phytochemicals in KOE for regulating activation of AMPK.

In summary, while DMC-2 in PMI-5011 improves glucose metabolism by enhancing insulin signaling in skeletal muscle, the compounds present in the KOE of PMI-5011 improve lipid metabolism in skeletal muscle by activating AMPK, a regulator of energy homeostasis. The multi-target “polypharmacological” paradigm of the complex mixture of phytochemicals found in PMI-5011 offers a strategy for treating the combined dysregulation of glucose and lipid metabolism characteristic of obesity-related metabolic diseases.

TABLE 1

| | | Concentration | |
|---|---|---|---|
| | Active compound isolated from | in % of | Name of |
| # | PMI-5011 | weight | fractions |
| 1 | 2′,4′-Dihydroxy-4- | 20.6 | Enriched |
| | methoxydihydrochalcone (DMC-2) | | DMC-2 |
| 2 | 2′,4-Dihydroxy-4′- | 11.1 | |
| | methoxydihydrochalcone (DMC-1) | | |
| 3 | Davidigenin | 7.9 | Knock- |
| 4 | 6-Demethoxycapillarisin | 19.0 | out |
| 5 | 4,5-di-O-caffeoylquinic acid | 41.3 | extract |
| | (Sakuranetin) | | (KOE) |
| 6 | Volatile compound (Elemicin) | <0.1 | |
| 7 | Volatile compound i(Iso-elemicin) | | |

### REFERENCES CITED IN THIS EXAMPLE

1. Hardie, D. G., Ross, F. A., and Hawley, S. A. (2012) AMPK: a nutrient and energy sensor that maintains energy homeostasis. Nat Rev Mol Cell Biol 13, 251-262
2. Hardie, D. G., Schaffer, B. E., and Brunet, A. (2016) AMPK: An Energy-Sensing Pathway with Multiple Inputs and Outputs. Trends Cell Biol 26, 190-201
3. Green, M. F., Anderson, K. A., and Means, A. R. (2011) Characterization of the CaMKKbeta-AMPK signaling complex. Cell Signal 23, 2005-2012
4. Garcia, D., and Shaw, R. J. (2017) AMPK: Mechanisms of Cellular Energy Sensing and Restoration of Metabolic Balance. Mol Cell 66, 789-800
5. Shulman, G. I., Rothman, D. L., Jue, T., Stein, P., DeFronzo, R. A., and Shulman, R. G. (1990) Quantitation of muscle glycogen synthesis in normal subjects and subjects with non-insulin-dependent diabetes by 13C nuclear magnetic resonance spectroscopy. N Engl J Med 322, 223-228
6. Brooks, G. A., and Mercier, J. (1994) Balance of carbohydrate and lipid utilization during exercise: the “crossover” concept. J Appl Physiol (1985) 76, 2253-2261
7. Dagenais, G. R., Tancredi, R. G., and Zierler, K. L. (1976) Free fatty acid oxidation by forearm muscle at rest, and evidence for an intramuscular lipid pool in the human forearm. J Clin Invest 58, 421-431
8. Witczak, C. A., Sharoff, C. G., and Goodyear, L. J. (2008) AMP-activated protein kinase in skeletal muscle: from structure and localization to its role as a master regulator of cellular metabolism. Cell Mol Life Sci 65, 3737-3755
9. Kelley, D. E. (2005) Skeletal muscle fat oxidation: timing and flexibility are everything. J Clin Invest 115, 1699-1702
10. Galgani, J. E., Moro, C., and Ravussin, E. (2008) Metabolic flexibility and insulin resistance. Am J Physiol Endocrinol Metab 295, E1009-1017
11. Samuel, V. T., and Shulman, G. I. (2016) The pathogenesis of insulin resistance: integrating signaling pathways and substrate flux. J Clin Invest 126, 12-22
12. Muoio, D. M. (2010) Intramuscular triacylglycerol and insulin resistance: guilty as charged or wrongly accused? Biochim Biophys Acta 1801, 281-288
13. Lowell, B. B., and Shulman, G. I. (2005) Mitochondrial dysfunction and type 2 diabetes. Science 307, 384-387
14. Witters, L. A. (2001) The blooming of the French lilac. J Clin Invest 108, 1105-1107
15. Shaw, R. J., Lamia, K. A., Vasquez, D., Koo, S. H., Bardeesy, N., Depinho, R. A., Montminy, M., and Cantley, L. C. (2005) The kinase LKB1 mediates glucose homeostasis in liver and therapeutic effects of metformin. Science 310, 1642-1646
16. Sakamoto, K., Goransson, O., Hardie, D. G., and Alessi, D. R. (2004) Activity of LKB1 and AMPK-related kinases in skeletal muscle: effects of contraction, phenformin, and AICAR. Am J Physiol Endocrinol Metab 287, E310-317
17. Hardie, D. G. (2006) Neither LKB1 nor AMPK are the direct targets of metformin. Gastroenterology 131, 973; author reply 974-975
18. Swanston-Flatt, S. K., Flatt, P. R., Day, C., and Bailey, C. J. (1991) Traditional dietary adjuncts for the treatment of diabetes mellitus. Proc Nutr Soc 50, 641-651
19. Kheterpal, I., Scherp, P., Kelley, L., Wang, Z., Johnson, W., Ribnicky, D., and Cefalu, W. T. (2014) Bioactives from Artemisia dracunculus L. enhance insulin sensitivity via modulation of skeletal muscle protein phosphorylation. Nutrition 30, S43-51
20. Vandanmagsar, B., Haynie, K. R., Wicks, S. E., Bermudez, E. M., Mendoza, T. M., Ribnicky, D., Cefalu, W. T., and Mynatt, R. L. (2014) Artemisia dracunculus L. extract ameliorates insulin sensitivity by attenuating inflammatory signalling in human skeletal muscle culture. Diabetes Obes Metab 16, 728-738
21. Kheterpal, I., Coleman, L., Ku, G., Wang, Z. Q., Ribnicky, D., and Cefalu, W. T. (2010) Regulation of insulin action by an extract of Artemisia dracunculus L. in primary human skeletal muscle culture: a proteomics approach. Phytother Res 24, 1278-1284
22. Logendra, S., Ribnicky, D. M., Yang, H., Poulev, A., Ma, J., Kennelly, E. J., and Raskin, I. (2006) Bioassay-guided isolation of aldose reductase inhibitors from Artemisia dracunculus. Phytochemistry 67, 1539-1546
23. Govorko, D., Logendra, S., Wang, Y., Esposito, D., Komarnytsky, S., Ribnicky, D., Poulev, A., Wang, Z., Cefalu, W. T., and Raskin, I. (2007) Polyphenolic compounds from Artemisia dracunculus L. inhibit PEPCK gene expression and gluconeogenesis in an H4IIE hepatoma cell line. Am J Physiol Endocrinol Metab 293, E1503-1510
24. Ribnicky, D. M., Poulev, A., Watford, M., Cefalu, W. T., and Raskin, I. (2006) Antihyperglycemic activity of Tarralin, an ethanolic extract of Artemisia dracunculus L. Phytomedicine 13, 550-557
25. Wang, Z. Q., Ribnicky, D., Zhang, X. H., Zuberi, A., Raskin, I., Yu, Y., and Cefalu, W. T. (2011) An extract of Artemisia dracunculus L. enhances insulin receptor signaling and modulates gene expression in skeletal muscle in KK-A(y) mice. J Nutr Biochem 22, 71-78
26. Yu, Y., Mendoza, T. M., Ribnicky, D. M., Poulev, A., Noland, R. C., Mynatt, R. L., Raskin, I., Cefalu, W. T., and Floyd, Z. E. (2018) An Extract of Russian Tarragon Prevents Obesity-Related Ectopic Lipid Accumulation. Mol Nutr Food Res 62, e1700856
27. Ribnicky, D. M., Kuhn, P., Poulev, A., Logendra, S., Zuberi, A., Cefalu, W. T., and Raskin, I. (2009) Improved absorption and bioactivity of active compounds from an anti-diabetic extract of Artemisia dracunculus L. Int J Pharm 370, 87-92
28. Yu, Y., Simmler, C., Kuhn, P., Poulev, A., Raskin, I., Ribnicky, D., Floyd, Z. E., and Pauli, G. F. (2019) The DESIGNER Approach Helps Decipher the Hypoglycemic Bioactive Principles of Artemisia dracunculus (Russian Tarragon). J Nat Prod 82, 3321-3329
29. Wang, Z. Q., Ribnicky, D., Zhang, X. H., Raskin, I., Yu, Y., and Cefalu, W. T. (2008) Bioactives of Artemisia dracunculus L enhance cellular insulin signaling in primary human skeletal muscle culture. Metabolism 57, S58-64
30. Fuller, S., Yu, Y., Mendoza, T., Ribnicky, D. M., Cefalu, W. T., and Floyd, Z. E. (2018) Potential adverse effects of botanical supplementation in high-fat-fed female mice. Biol Sex Differ 9, 41
31. Ribnicky, D. M., Poulev, A., Watford, M., Cefalu, W. T., and Raskin, I. (2006) Antihyperglycemic activity of Tarralin, an ethanolic extract of Artemisia dracunculus L. Phytomedicine 13, 550-557
32. Ribnicky, D. M., Kuhn, P., Poulev, A., Logendra, S., Zuberi, A., Cefalu, W. T., and Raskin, I. (2009) Improved absorption and bioactivity of active compounds from an anti-diabetic extract of Artemisia dracunculus L. Int J Pharm 370, 87-92
33. Zuberi, A. R. (2008) Strategies for assessment of botanical action on metabolic syndrome in the mouse and evidence for a genotype-specific effect of Russian tarragon in the regulation of insulin sensitivity. Metabolism 57, S10-15
34. Schmidt, B., Ribnicky, D. M., Poulev, A., Logendra, S., Cefalu, W. T., and Raskin, I. (2008) A natural history of botanical therapeutics. Metabolism 57, S3-9
35. Reddy, A. S., and Zhang, S. (2013) Polypharmacology: drug discovery for the future. Expert Review of Clinical Pharmacology 6, 1-13
36. Zhu, H., Moriasi, C. M., Zhang, M., Zhao, Y., and Zou, M. H. (2013) Phosphorylation of serine 399 in LKB1 protein short form by protein kinase Czeta is required for its nucleocytoplasmic transport and consequent AMP-activated protein kinase (AMPK) activation. J Biol Chem 288, 16495-16505
37. Jeon, S. M. (2016) Regulation and function of AMPK in physiology and diseases. Exp Mol Med 48, e245
38. Racioppi, L., and Means, A. R. (2012) Calcium/calmodulin-dependent protein kinase kinase 2: roles in signaling and pathophysiology. J Biol Chem 287, 31658-31665
39. York, B., Li, F., Lin, F., Marcelo, K. L., Mao, J., Dean, A., Gonzales, N., Gooden, D., Maity, S., Coarfa, C., Putluri, N., and Means, A. R. (2017) Pharmacological inhibition of CaMKK2 with the selective antagonist STO-609 regresses NAFLD. Sci Rep 7, 11793
40. Marcelo, K. L., Means, A. R., and York, B. (2016) The Ca(2+)/Calmodulin/CaMKK2 Axis: Nature's Metabolic CaMshaft. Trends Endocrinol Metab 27, 706-718
41. Ruderman, N. B., Saha, A. K., and Kraegen, E. W. (2003) Minireview: Malonyl CoA, AMP-Activated Protein Kinase, and Adiposity. Endocrinology 144, 5166-5171
42. Canto, C., Gerhart-Hines, Z., Feige, J. N., Lagouge, M., Noriega, L., Milne, J. C., Elliott, P. J., Puigserver, P., and Auwerx, J. (2009) AMPK regulates energy expenditure by modulating NAD+ metabolism and SIRT1 activity. Nature 458, 1056-1060
43. Cuyas, E., Verdura, S., Llorach-Pares, L., Fernandez-Arroyo, S., Joven, J., Martin-Castillo, B., Bosch-Barrera, J., Brunet, J., Nonell-Canals, A., Sanchez-Martinez, M., and Menendez, J. A. (2018) Metformin Is a Direct SIRT1-Activating Compound: Computational Modeling and Experimental Validation. Front Endocrinol (Lausanne) 9, 657
44. Hardie, D. G. (2013) AMPK: a target for drugs and natural products with effects on both diabetes and cancer. Diabetes 62, 2164-2172
45. Mounier, R., Lantier, L., Leclerc, J., Sotiropoulos, A., Foretz, M., and Viollet, B. (2011) Antagonistic control of muscle cell size by AMPK and mTORC1. Cell Cycle 10, 2640-2646
46. Miyamoto, T., Rho, E., Sample, V., Akano, H., Magari, M., Ueno, T., Gorshkov, K., Chen, M., Tokumitsu, H., Zhang, J., and Inoue, T. (2015) Compartmentalized AMPK signaling illuminated by genetically encoded molecular sensors and actuators. Cell Rep 11, 657-670
47. Zong, Y., Zhang, C. S., Li, M., Wang, W., Wang, Z., Hawley, S. A., Ma, T., Feng, J. W., Tian, X., Qi, Q., Wu, Y. Q., Zhang, C., Ye, Z., Lin, S. Y., Piao, H. L., Hardie, D. G., and Lin, S. C. (2019) Hierarchical activation of compait nentalized pools of AMPK depends on severity of nutrient or energy stress. Cell Res 29, 460-473
48. Salt, I., Celler, J. W., Hawley, S. A., Prescott, A., Woods, A., Carling, D., and Hardie, D. G. (1998) AMP-activated protein kinase: greater AMP dependence, and preferential nuclear localization, of complexes containing the alpha2 isoform. Biochem J 334 (Pt 1), 177-187
49. Ronnebaum, S. M., Patterson, C., and Schisler, J. C. (2014) Minireview: Hey U(PS): Metabolic and Proteolytic Homeostasis Linked via AMPK and the Ubiquitin Proteasome System. Molecular Endocrinology 28, 1602-1615
50. Zungu, M., Schisler, J. C., Essop, M. F., McCudden, C., Patterson, C., and Willis, M. S. (2011) Regulation of AMPK by the ubiquitin proteasome system. Am J Pathol 178, 4-11
51. Treebak, J. T., Glund, S., Deshmukh, A., Klein, D. K., Long, Y. C., Jensen, T. E., Jorgensen, S. B., Viollet, B., Andersson, L., Neumann, D., Wallimann, T., Richter, E. A., Chibalin, A. V., Zierath, J. R., and Wojtaszewski, J. F. (2006) AMPK-mediated AS160 phosphorylation in skeletal muscle is dependent on AMPK catalytic and regulatory subunits. Diabetes 55, 2051-2058
52. Lantier, L., Williams, A. S., Williams, I. M., Guerin, A., Bracy, D. P., Goelzer, M., Foretz, M., Viollet, B., Hughey, C. C., and Wasserman, D. H. (2020) Reciprocity Between Skeletal Muscle AMPK Deletion and Insulin Action in Diet-Induced Obese Mice. Diabetes 69, 1636-1649
53. Samovski, D., Sun, J., Pietka, T., Gross, R. W., Eckel, R. H., Su, X., Stahl, P. D., and Abumrad, N. A. (2015) Regulation of AMPK activation by CD36 links fatty acid uptake to beta-oxidation. Diabetes 64, 353-359
54. Canto, C., Jiang, L. Q., Deshmukh, A. S., Mataki, C., Coste, A., Lagouge, M., Zierath, J. R., and Auwerx, J. (2010) Interdependence of AMPK and SIRT1 for Metabolic Adaptation to Fasting and Exercise in Skeletal Muscle. Cell Metabolism 11, 213-219
55. Xie, Z., Dong, Y., Scholz, R., Neumann, D., and Zou, M. H. (2008) Phosphorylation of LKB1 at serine 428 by protein kinase C-zeta is required for metformin-enhanced activation of the AMP-activated protein kinase in endothelial cells. Circulation 117, 952-962
56. Luna, V., Casauban, L., Sajan, M. P., Gomez-Daspet, J., Powe, J. L., Miura, A., Rivas, J., Standaert, M. L., and Farese, R. V. (2006) Metformin improves atypical protein kinase C activation by insulin and phosphatidylinositol-3,4,5-(PO4)3 in muscle of diabetic subjects. Diabetologia 49, 375-382
57. Li, S., Zhang, Y., Sun, Y., Zhang, G., Bai, J., Guo, J., Su, X., Du, H., Cao, X., Yang, J., and Wang, T. (2019) Naringenin improves insulin sensitivity in gestational diabetes mellitus mice through AMPK. Nutr Diabetes 9, 28
58. Zygmunt, K., Faubert, B., MacNeil, J., and Tsiani, E. (2010) Naringenin, a citrus flavonoid, increases muscle cell glucose uptake via AMPK. Biochem Biophys Res Commun 398, 178-183
59. Yu, L. M., Dong, X., Xue, X. D., Zhang, J., Li, Z., Wu, H. J., Yang, Z. L., Yang, Y., and Wang, H. S. (2019) Naringenin improves mitochondrial function and reduces cardiac damage following ischemia-reperfusion injury: the role of the AMPK-SIRT3 signaling pathway. Food Funct 10, 2752-2765
60. Mutlur Krishnamoorthy, R., and Carani Venkatraman, A. (2017) Polyphenols activate energy sensing network in insulin resistant models. Chemico-Biological Interactions 275, 95-107
61. Mo, Z., Li, L., Yu, H., Wu, Y., and Li, H. (2019) Coumarins ameliorate diabetogenic action of dexamethasone via Akt activation and AMPK signaling in skeletal muscle. J Pharmacol Sci 139, 151-157
62. Jang, J. H., Park, J. E., and Han, J. S. (2020) Scopoletin increases glucose uptake through activation of PI3K and AMPK signaling pathway and improves insulin sensitivity in 3T3-L1 cells. Nutr Res 74, 52-61

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A method of reducing blood glucose levels, the method comprising administering to a subject in need thereof a therapeutically effective amount of a composition comprising one or more components of PMI-5011.

2. A method of modulating glucose homeostasis, the method comprising administering to a subject in need thereof a therapeutically effective amount of a composition comprising one or more components of PMI-5011.

3. A method of treating diabetes, the method comprising administering to a subject in need thereof a therapeutically effective amount of a composition comprising one or more components of PMI-5011.

4. The method of claim 1, 2, or 3, wherein the one or more components comprise a methyldavidigenin.

5. The method of claim 4, wherein the methyldavidigenin comprises 2′,4′-dihydroxy-4-methoxydihydrochalcone (DMC-2), 4,2′-dihydroxy-4′-methoxydihydrochalcone (DMC-1), or both.

6. The method of claim 1, 2 or 3, wherein the one or more components comprise DMC-2, DMC-1, elemicin, sakuranetin, davidigenin, 6-demethoxycapillarisin or any combination thereof.

7. The method of claim 1, 2 or 3, wherein the composition does not contain DMC-1, DMC-2, or both.

8. The method of claim 1, 2, or 3, wherein the composition comprises reduced levels of DMC-1, DMC-2, or both.

9. The method of claim 1, 2 or 3, wherein the components are naturally derived, synthetically produced, or a combination thereof.

10. The method of claim 1, 2 or 3, wherein the composition is formulated for oral administration.
